(19) United States
(12) Patent Application Publication
(10) Pub. No.: US 2025/0037817 A1
(43) Pub. Date: Jan. 30, 2025

(54) RISK ADJUSTED MORTALITY RATE USING AUTOMATED DETERMINATION OF PATIENT CO-MORBIDITIES

(71) Applicant: THE RESEARCH FOUNDATION FOR THE STATE UNIVERSITY OF NEW YORK, Albany, NY (US)

(72) Inventors: I.V. Ramakrishnan, Setauket, NY (US); Mark Henry, Northport, NY (US)

(21) Appl. No.: 18/916,203

(22) Filed: Oct. 15, 2024

Related U.S. Application Data
(63) Continuation of application No. 16/535,687, filed on Aug. 8, 2019, now abandoned.

(51) Int. Cl. G16H 10/60; G06F 16/33; G16H 15/00; G16H 50/30

(52) U.S. Cl. CPC G16H 10/60; G06F 16/3344; G16H 15/00; G16H 50/30

(57) ABSTRACT

Systems and methods for decreasing risk-adjusted mortality rates, including identifying patient co-morbidity-related clinical data by preprocessing historical patient data based on rules. Identified co-morbidity-related clinical data is displayed using an interactive user interface for validation, and validated results are stored in an electronic health record (EHR) for the patient. Updated co-morbidity-related clinical is identified by concurrent processing updated patient data based on the rules. The updated co-morbidity-related clinical data is displayed using the interactive user interface for further validation. The EHR is iteratively updated with revised co-morbidity data by the postprocessing upon detection of changes to the EHR.

### CROSS REFERENCE TO RELATED APPLICATIONS

This application is a Continuation of U.S. patent application Ser. No. 16/535,687 filed on Aug. 19, 2019.

### BACKGROUND

### Technical Field

The present invention relates to automated determination and decision support of patient's co-morbid conditions (co-morbidities), and more particularly to improving hospital risk adjusted metrics (e.g., mortality rates, etc.) by accurately and efficiently identifying a patient's co-morbid conditions (co-morbidities) using electronic health records (EHRs) in real-time.

### Description of the Related Art

Health Systems across the globe are focused on moving to electronic health records (EHRs) both in the hospital and ambulatory settings to make patients' clinical information easily retrievable across settings. In addition, the federal government has tied reimbursement for hospitals and physicians to demonstration of meaningful use of EHRs. As the use of EHRs is relatively new and its potential great, there are multiple opportunities to improve the products in current use. Improvements to the EHR that can streamline the ease of use and push valuable information to the provider during the episode of care can both increase the quality of health care provided as well as enhance accurate reimbursement are needed and will find widespread application and adoption.

Co-morbidities, or comorbid conditions, are significant medical conditions that impact on a patient's health, and yet are not the principle or primary diagnosis or reason for a patient encounter with medical services. Co-morbid conditions can impact a patient's current care as well as affect a patients' healing, survival and length of hospitalization. Knowledge and documentation of co-morbidities is important information for a patient's health team for proper patient treatment. Moreover, proper documentation of co-morbidities is important for hospital coding in analyzing service intensity weight, risk adjusted outcome measures, staffing and reimbursement.

### SUMMARY

A method for decreasing risk-adjusted mortality rates, including identifying patient co-morbidity-related clinical data by preprocessing historical patient data based on rules. Identified co-morbidity-related clinical data is displayed using an interactive user interface for validation, and validated results are stored in an electronic health record (EHR) for the patient. Updated co-morbidity-related clinical is identified by concurrent processing updated patient data based on the rules. The updated co-morbidity-related clinical data is displayed using the interactive user interface for further validation. The EHR is iteratively updated with revised co-morbidity data by the concurrent processing upon detection of changes to the EHR.

A system for decreasing risk-adjusted mortality rates, including identifying, using a processor operatively coupled to a non-transitory computer readable storage medium, patient co-morbidity-related clinical data by preprocessing historical patient data based on rules. Identified co-morbidity-related clinical data is displayed using an interactive user interface for validation, and validated results are stored in an electronic health record (EHR) for the patient. Updated co-morbidity-related clinical is identified by concurrent processing updated patient data based on the rules. The updated co-morbidity-related clinical data is displayed using the interactive user interface for further validation. The EHR is iteratively updated with revised co-morbidity data by the concurrent processing upon detection of changes to the EHR.

A non-transitory computer readable storage medium including contents that are configured to cause a computer to perform a method for decreasing risk-adjusted mortality rates, the method including identifying patient co-morbidity-related clinical data by preprocessing historical patient data based on rules. Identified co-morbidity-related clinical data is displayed using an interactive user interface for validation, and validated results are stored in an electronic health record (EHR) for the patient. Updated co-morbidity-related clinical is identified by concurrent processing updated patient data based on the rules. The updated co-morbidity-related clinical data is displayed using the interactive user interface for further validation. The EHR is iteratively updated with revised co-morbidity data by the concurrent processing upon detection of changes to the EHR.

These and other features and advantages will become apparent from the following detailed description of illustrative embodiments thereof, which is to be read in connection with the accompanying drawings.

### BRIEF DESCRIPTION OF THE DRAWINGS

The disclosure will provide details in the following description of preferred embodiments with reference to the following figures wherein:

FIG. 1 shows an exemplary processing system to which the present principles may be applied, in accordance with an embodiment of the present invention;

FIG. 2 is a block/flow diagram showing an exemplary high-level system architecture for automated determination, analysis, and/or presentation of patient co-morbidities, in accordance with an embodiment of the present invention;

FIG. 3 is a diagram showing an exemplary display of a clinician's view of a patient's information, in accordance with an embodiment of the present invention;

FIG. 4 is a diagram showing an exemplary display of an emergency room diagnosis (ED) checklist of co-morbidity, in accordance with an embodiment of the present invention;

FIG. 5 is a block/flow diagram showing an exemplary high-level system architecture for automated determination, analysis, and/or presentation of patient co-morbidities, in accordance with an embodiment of the present invention;

FIG. 6A is a diagram showing an exemplary customized graphical user interface (GUI) display screen, in accordance with an embodiment of the present invention;

FIG. 6B is a diagram showing an exemplary customized graphical user interface (GUI) display screen, in accordance with an embodiment of the present invention;

FIG. 6C is a diagram showing an exemplary customized graphical user interface (GUI) display screen, in accordance with an embodiment of the present invention;

FIG. 6D is a diagram showing an exemplary customized graphical user interface (GUI) display screen, in accordance with an embodiment of the present invention;

FIG. 6E is a diagram showing an exemplary customized graphical user interface (GUI) display screen, in accordance with an embodiment of the present invention;

FIG. 6F is a diagram showing an exemplary customized graphical user interface (GUI) display screen, in accordance with an embodiment of the present invention;

FIG. 6G is a diagram showing an exemplary customized graphical user interface (GUI) display screen, in accordance with an embodiment of the present invention;

FIG. 6H is a diagram showing an exemplary customized graphical user interface (GUI) display screen, in accordance with an embodiment of the present invention;

FIG. 6I is a diagram showing an exemplary customized graphical user interface (GUI) display screen, in accordance with an embodiment of the present invention;

FIG. 6J is a diagram showing an exemplary customized graphical user interface (GUI) display screen, in accordance with an embodiment of the present invention;

FIG. 6K is a diagram showing an exemplary customized graphical user interface (GUI) display screen, in accordance with an embodiment of the present invention;

FIG. 6L is a diagram showing an exemplary customized graphical user interface (GUI) display screen, in accordance with an embodiment of the present invention;

FIG. 6M is a diagram showing an exemplary customized graphical user interface (GUI) display screen, in accordance with an embodiment of the present invention;

FIG. 6N is a diagram showing an exemplary customized graphical user interface (GUI) display screen, in accordance with an embodiment of the present invention;

FIG. 7 is a block/flow diagram showing a method for automated identification,

documentation, and displaying of co-morbidities from patient Electronic Health Records (EHRs), in accordance with an embodiment of the present invention;

FIG. 8 is a block/flow diagram showing a method for automated determination, analysis, and/or presentation of patient co-morbidities, in accordance with an embodiment of the present invention; and

FIG. 9 is a block diagram showing a system for automated determination, analysis, and/or presentation of patient co-morbidities, in accordance with an embodiment of the present invention.

### DETAILED DESCRIPTION

In accordance with the present invention, systems and methods are provided for automated determination, analysis, and/or presentation of patient co-morbidities based on clinical data found in the electronic health records (EHRs) in the form of structured and unstructured data. In accordance with various embodiments, a novel system and method to automatically identify and document co-morbidities, or comorbid conditions, from patient's EHR is presented. The system can be used within health IT systems to automatically search EHRs and determine comorbid conditions in a timely and accurate manner for review, validation and subsequent documentation by physicians. It enhances patient care, more accurately documents patient illness, and assures proper reimbursement.

During a patient encounter in the emergency department of a hospital or care center, there can be an opportunity to document what co-morbidities a patient may have, so that care givers are aware and can address them. Often these co-morbidities, which may be discovered during the encounter, are not the principle reason for the encounter or visit. Thus, these co-morbidities may not be listed as a diagnosis or reason for visit in the record, even though the co-morbidities may affect the severity of the illness that brought the patient to the emergency department. For accurate coding, reimbursement and data collection purposes, the co-morbidities must be expressed in the narrative description (such as hyperpotassemia when the K+ is 6.5) and signed by the physician. At times, a comorbid condition may be categorized as secondary diagnosis, such as acute respiratory failure when the principle diagnosis is pneumonia. Such a secondary diagnosis can impact any of a plurality of health-care related events, diagnoses, treatments, etc. (e.g., a patient's risk of mortality, length of hospitalization, reimbursement to both the provider and the hospital, etc. However, none of this is realized without proper documentation that this comorbid condition exists even though it has impacted the patients current care and the provider's medical decision making.

Electronic health records (EHRs) should improve identification and documentation of pertinent health issues. They should be faster and better than paper records. However, many Computerized Physician Order Entry (CPOE) and EHRs today result in physicians and nurses spending more time at the computer than at the bedside because data collection and entry, not patient care, becomes the focus, resulting in inefficiency and inaccuracy. With large amounts of data input to the EHR from multiple healthcare practitioners, there is the danger of data overload, resulting in the need to cull through extraneous information to find that which is pertinent. As these data entry tasks take time from the doctor-patient encounter, the tasks are often ignored and/or left incomplete because of time constraints.

For example, with respect to co-morbidities, existing methods for identifying co-morbidities data in EHRs have relied primarily on costly and time-consuming manual chart review, and thus is incomplete and/or inaccurate for real-time (e.g., hospital) use. During a visit to the ER, voluminous amounts of information become available during the work-up but because immediate treatment is being focused on the principal diagnosis some of the co-morbid conditions are often missed. As a result, accidental omissions and misdiagnosis can often occur in practice. Moreover, in certain cases the physician may incorrectly decide to ignore particular co-morbidities as an unimportant or a transient occurrence at least in part because of such inaccuracies. The physician may not take the time to list conditions that were present at the time of presentation and admission simply because of the amount of overwhelming data in the EHR and the inability to remember what important comorbid conditions are affecting this patient's current risk of morbidity and mortality. Failure to document these comorbid conditions in the medical record leads to poor communication to the other providers in the care team and can lead to lack of coordination of patient care, increased lengths of stays, decreased revenue and unfortunately, poorer outcomes.

In one aspect, the method can comprise selecting co-morbidity-related clinical data in accordance with one or more rules, the clinical data selected for a patient from the history data of the patient, pushing the selected clinical data to a display on a user interface, displaying the selected clinical data on the display along with the one or more rules, analyzing the displayed selected clinical data in accordance with the displayed one or more rules, validating the displayed selected data, and storing the validated data as part of the medical records.

A novel system and method and computer program for identifying and documenting co-morbidities is presented. In one aspect, the innovative technology automatically searches a patient's EHR and extracts co-morbidities (“co-morbidity-related clinical data”) from laboratory values, vital signs monitoring, radiology reports, and other electronic data fields (such as medication lists), and lists these co-morbidities on a co-morbidity display for review and validation by the attending physician. The co-morbidities can be current clinical conditions. The inventive system presented herein differs from prior systems in several ways.

For example, the novel technology works on EHRs in real-time and is fully automatic. It simplifies and speeds documentation by decreasing the data entry burden on clinicians as it pushes information from current and past health care encounters to the provider for validation and approval of automated entry. Also, the inventive technology handles a variety of co-morbidities in a general patient population, as opposed to systems that handle only one type of patients, such as cancer patients.

In various embodiments, the present invention can process historical and current documented clinical findings, using proven medical algorithms to indicate the presence of clinical conditions which appear to exist in the patient. These conditions can be identified according to definitions promulgated by CMS and/or authoritative professional society by specific names which convey relevance to clinicians as either co-morbid conditions (CCs) or major complicating conditions (MCCs) which might impact upon the primary pathologic condition(s) which have caused the patient to present for evaluation. Conditions referred to here are actually factual items. (e.g. elevated chemical values like potassium or sodium levels, a positive physical diagnosis such as a pneumonia on x-ray or heart attack by EKG, etc.). The condition may be either a single factor or a cluster of factors, but they are factual, actual and not calculated. Rather they are collected as a compendium. The clinician can employ training, knowledge and judgment on each presented item to determine the relevance of the condition to the patient in real-time (e.g., during patient interaction), or at any time in accordance with various embodiments.

While other Clinical Document Improvement (CDI) methods concentrate on analysis and correction of written clinical documentation as a retrospective basis of analysis (e.g., after a patient has been admitted to, treated, or discharged from a hospital), the present invention can utilize the raw data from clinical reporting systems to inform the clinician of previously defined conditions before final impressions are recorded for the clinical interaction, in accordance with various embodiments of the present invention.

A purpose of Clinical Document improvement systems (CDI) is to create the most

accurate description of the patient possible. the difference is in the method employed to approach that result. The current state of the art is to post process or correct the observations that have been documented by the clinician after they have been recorded. The clinician's work can be reviewed, by either clinical documentation specialists, software, or some combination to determine whether there might be other factors that should be added to the original work to make it more precise. Then, queries (e.g., questions) are presented to the clinician who made the original notations which encourage review and possible revision of what has already been they have documented. This process is roughly analogous to spell or grammar checking a document after it has been written. There are several problems with this approach. It can be very time consuming for the clinician who still has to review the work already done, sometimes more than once. It also increases the risk that an outside reviewer will consider the activity as an attempt to “upcode” or artificially increase the severity of that patient's illness, retrospectively, to increase billings. In addition, it requires added rework for the physician which impinges on their work of the moment.

Preprocessing, in accordance with various embodiments of the present invention, can, at the time that the clinician is documenting the patient's findings in the record, examine the electronic medical record of that patient in real-time to find current relevant clinical indicators and conditions that have been documented in the past. This compendium of findings can be presented for inclusion, if they are deemed to be relevant to the current situation. The preprocessing can efficiently and accurately acquire and review all of the clinical indicators in the EHR documentation concurrently with starting to care for the patient, and preprocessing is much less time consuming, more efficient and can further be dependent on the clinical judgments of the moment. It does not require rework and is therefore much more acceptable to busy clinicians than any previous systems and methods.

In some embodiments, data on the patient can be analyzed, sequentially testing its discovery definitions against each data item defined in its algorithms. The present invention can utilize Natural Language Processing (NLP) to extract relevant target data from text, or any sort of structured or unstructured data records in accordance with various embodiments. When a targeted diagnosis or data point is found, it can be collected, pre-defined clusters of data and diagnoses can be assembled, according to rules, and held for display.

In some embodiments, time interval search parameters can be adjusted, and the invention can function as a new type of concurrent processor, picking up new clinical data points that have been added to the patient record after the initial pre-processing run. In this manner, the present invention can improve the current diagnosis and EHRs, and can be viewed an adjunct to CDI and coding teams. The present invention enables a selection of start and ending dates (e.g., the start can be set at a date certain and then run forward), and this enables tuning of later notes and observations after the initial use, in accordance with various embodiments of the present invention.

In accordance with various embodiments of the present invention, demonstrable effects (e.g., experimental results) on the generation of much more accurate data and thus diagnosis, have been found, which, when used over time lead to better epidemiological data. Since this data is used by CMS to set quality and reimbursement relationships, corrected discovery leads to more accuracy in Observed to Expected (O/E) mortality rates, Case Mix Indices (CMI) and the quality and/or reimbursement rates that are derived from those measures.

Comorbid conditions (comorbidities) are medical problems and pathological processes that occur in relation to a primary disease state [Def-1]. When patients present to the Emergency Department, comorbidities can be unrelated to the pathological process that is the chief medical complaint but can affect the complexity of treatment and even the outcome for that patient.

Comorbidities can affect a patients' healing, survival, and length of hospitalization. Moreover, proper identification and documentation of comorbid conditions are important on many levels. Comorbid conditions that are recognized and documented in the Emergency Department or during the inpatient hospital stay increase the service intensity weight thereby increasing the reimbursement for that patient as well as their expected length of stay. Documented comorbid conditions, along with the patient's age, race, discharge status and procedures performed become part of coded administrative data. This becomes the data that private insurance and government agencies use to calculate risk-adjusted mortality, morbidity and more recently, risk adjusted readmission rates for that patient.

The ratio between actual and expected death of a patient, adjusted in risk models, is now considered a reflection on quality of care and will be one of the quality measures that Medicare and Medicaid base hospital and physician reimbursement on. There are several sources of risk models that are formed using year-to-year logistic regression analysis of diagnostic related groups and their comorbid conditions. All risk models, whether they come from The Agency of Health Related Quality (AHRQ), the federal government or a private agency, utilize the principal diagnosis of the patient along with demographics and the interactions of the patient's comorbid conditions to determine that patient's current severity of illness and their risk of mortality. The comorbid conditions used in the risk model need to be present prior to the patient's admission to a hospital in order to be applied to a patient's risk adjustment. Conditions that occur after the patient's admission may be considered hospital acquired and may reflect negatively on the quality of care for the hospital and the physician taking care of that patient.

Each risk model, determined by the patient's principal diagnosis, has comorbid conditions in which there is a coefficient relating that comorbidity to the likelihood of death. Although risk adjustment may appear to be a sound method of examining hospital and physician mortality rates, there are significant problems identified with these models. One of the major problems is that documentation of the conditions must be in the medical record in order for the professional coder to code the diagnosis so that it will become part of the administrative data submitted for this patient. Under-documentation can occur because physicians do not recognize specific comorbidities that present as a mortality risk for a specific diagnostic related group. This leaves hospitals and physicians to appear to care for healthier patients than presented in data (Shine, D., 2012). There is much written in the literature regarding utilization of risk adjusted data such as mortality rates as a quality of care indicator. It has been found that under reported comorbidities and miscoded data has caused measurement error that range from 0.2 to 2.2 expected 30-day deaths per 100 admissions. Other studies have shown that without the inclusion and proper identification of comorbid conditions as being present at the time of admission to the hospital, the quality ranking of hospital may be inappropriately reported. Inclusion of a present on admission indicator for a comorbid condition frequently changes the quality ranking of hospitals classified as low quality to high quality.

Without proper identification of conditions that are preexisting at the time of admission to the hospital, it is impossible to differentiate preexisting conditions from complications that occur after hospitalization due to quality of care. This differentiation is important to ensure unbiased risk adjustment of patients and accurately reported administrative data and quality outcomes. A study done in 2009 strongly supports the value of increasing the consistency of reporting International Classification of Diseases, Tenth Revision, Clinical Modification (ICD-10-CM) codes of secondary diagnoses that are important predictors of hospital outcomes as well as adding laboratory results which significantly improved predictions of a patient's mortality. As risk adjusted mortality starts to become one of the key quality indicators that will affect hospital and physician payment in the future, more and more studies are being done to look at some of the top comorbid conditions that affect a specific type of patient's risk of mortality. A more recent study in 2012, identified the most common comorbid conditions across risk models used by 3M (as used by CMS) and UHC.

In various embodiments, the present invention can enable the emergency room physician to run the program inside the electronic medical record prior to the patient's admission and pulls together any vital signs, labs or radiology values from the patient's current encounter or in some instances, past encounters. Significant criteria found by the software are pushed to the ER physician along with the recommended comorbid conditions that most strongly correlates to that criterion. The physician can then be asked to evaluate the recommended comorbid condition and the clinical indicators that have been found in the medical record and validate them. The system can then automatically populate the diagnosis in the medical record, which is then easily accessible by both hospital data abstractors and hospital professional coders. Automatic push of validated co-morbidities in accordance with embodiments of the present invention improves accuracy, and saves the clinician time and eliminate documentation omissions and errors while automated population of diagnosis to the medical record greatly reduces the possibility chance that important diagnoses may be missed (e.g., by clinicians, data abstractors, coders, etc.). As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects. Furthermore, embodiments of the present invention may take the form of a computer program product embodied in one or more computer readable medium(s) having computer readable program code embodied thereon.

Any combination of one or more computer readable medium(s) may be employed. The computer readable medium may be a computer readable signal medium or a computer readable storage medium. A computer readable storage medium may be, but is not limited to, an electronic, magnetic, optical, electromagnetic, infrared, or semiconductor system, apparatus, or device, or any combination thereof. Other examples of the computer readable storage medium may include, but are not limited to, an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any combination thereof. In this document, a computer readable storage medium may be any tangible medium that can contain, or store a program for use by or in connection with a computing system, apparatus, or device.

Program code embodied on a computer readable medium may be transmitted using any suitable medium, including but not limited to wireless, wireline, optical fiber cable, etc., or any combination thereof. Computer program code for carrying out operations for aspects of the present invention may be written in any combination of one or more programming languages, including, but not limited to any general-purpose programing language (e.g., PHP, Java, C++, etc.) and/or domain-specific programing language (e.g. HTML, SQL, etc.). The program code may execute fully on the user's computer/mobile device, partially on the user's computer/mobile device, as stand-alone software, partially on the user's computer/mobile device and partially on a remote computer/mobile device, or entirely on a remote computer or server. The remote computer may be connected to the user's computer through any type of network (e.g., a local area network (LAN), wide area network (WAN), a connection to an external computer (e.g., over the Internet using an Internet Service Provider), etc.).

Aspects of the present invention are described below with reference to flowchart illustrations and/or block diagrams of methods, systems and computer program products according to embodiments of the present invention. It is noted that each block of the flowcharts and/or block diagrams, and combinations of blocks in the flowcharts and/or block diagrams, may be implemented by computer program instructions.

These computer program instructions may be sent to a processor of any type of computing system (e.g., general purpose computer, special purpose computer, or other programmable data processing apparatus to produce a machine), such that the instructions, which execute by the processor of the computing system, create a means for implementing the functions/instructions/acts specified in the flowcharts and/or block diagram block or blocks. These computer program instructions may also be stored in a computer readable medium that can instruct any computing device to function in a particular manner, such that the instructions stored in the computer readable medium produce an article of manufacture including instructions which implement the function/instruction/act specified in the flowchart and/or block diagram block or blocks.

The computer program instructions may also be loaded onto a computer, mobile device, other programmable data processing apparatus, or other devices to cause a series of operational steps to be performed on any computing system to produce a computer implemented process such that the instructions which execute on the computer or other programmable apparatus provide processes for implementing the functions/acts specified in the flowcharts and/or block diagram block or blocks.

A computer readable signal medium may include a propagated data signal with computer readable program code embodied therein (e.g., baseband, part of a carrier wave, etc.). Such a propagated signal may take any of a variety of forms, including, but not limited to, electro-magnetic, optical, or any combination thereof. A computer readable signal medium may be any computer readable medium that is not a computer readable storage medium and that can communicate, propagate, or transport a program for use by or in connection with a computing system, apparatus, or device.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

The flowchart and block diagrams in the figures illustrate the architecture, functionality, and operation of possible implementations of systems, methods and computer program products according to various embodiments of the present invention. Each block in the flowchart or block diagrams may represent a module, segment, or portion of code, which comprises one or more executable instructions for implementing the specified logical function(s), and in some alternative implementations of the present invention, the functions noted in the block may occur out of the order noted in the figures. For example, two blocks shown in succession may, in fact, be executed substantially concurrently, may sometimes be executed in reverse order, or may be executed in any other order, depending on the functionality of a particular embodiment.

It is also noted that each block of the block diagrams and/or flowchart illustration, and combinations of blocks in the block diagrams and/or flowchart illustration, can be implemented by specific purpose hardware systems that perform the specific functions/acts, or combinations of special purpose hardware and computer instructions according to the present principles.

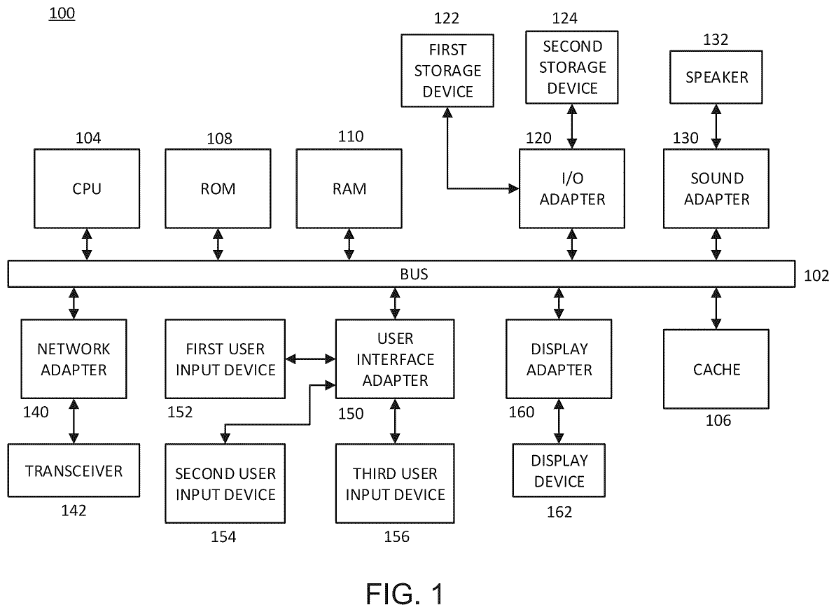

Referring now to the drawings in which like numerals represent the same or similar elements and initially to FIG. 1, an exemplary processing system 100, to which the present principles may be applied, is illustratively depicted in accordance with an embodiment of the present principles. The processing system 100 includes at least one processor (CPU) 104 operatively coupled to other components via a system bus 102. A cache 106, a Read Only Memory (ROM) 108, a Random Access Memory (RAM) 110, an input/output (I/O) adapter 120, a sound adapter 130, a network adapter 140, a user interface adapter 150, and a display adapter 160, are operatively coupled to the system bus 102.

A first storage device 122 and a second storage device 124 are operatively coupled to system bus 102 by the I/O adapter 120. The storage devices 122 and 124 can be any of a disk storage device (e.g., a magnetic or optical disk storage device), a solid state magnetic device, and so forth. The storage devices 122 and 124 can be the same type of storage device or different types of storage devices.

A speaker 132 is operatively coupled to system bus 102 by the sound adapter 130. A transceiver 142 is operatively coupled to system bus 102 by network adapter 140. A display device 162 is operatively coupled to system bus 102 by display adapter 160.

A first user input device 152, a second user input device 154, and a third user input device 156 are operatively coupled to system bus 102 by user interface adapter 150. The user input devices 152, 154, and 156 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present principles. The user input devices 152, 154, and 156 can be the same type of user input device or different types of user input devices. The user input devices 152, 154, and 156 are used to input and output information to and from system 100.

Of course, the processing system 100 may also include other elements (not shown), as readily contemplated by one of skill in the art, as well as omit certain elements. For example, various other input devices and/or output devices can be included in processing system 100, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 100 are readily contemplated by one of ordinary skill in the art given the teachings of the present principles provided herein.

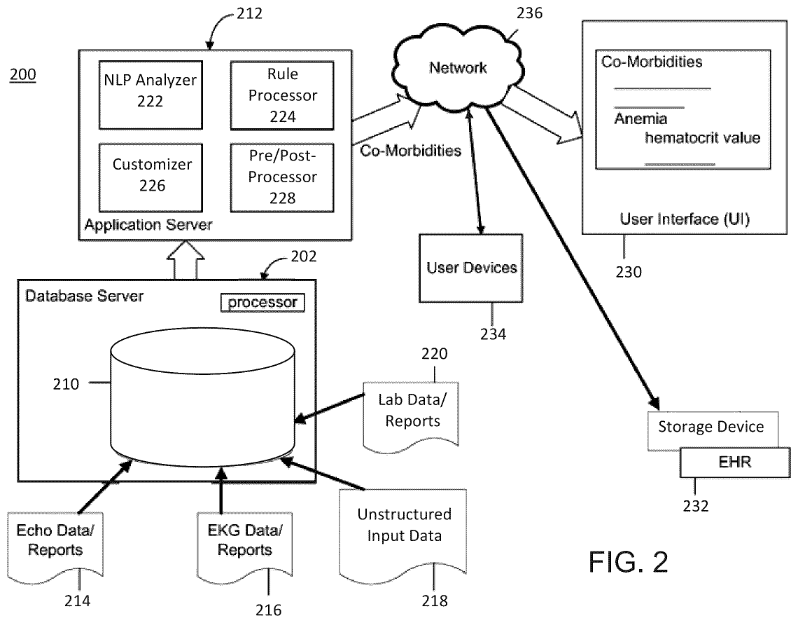

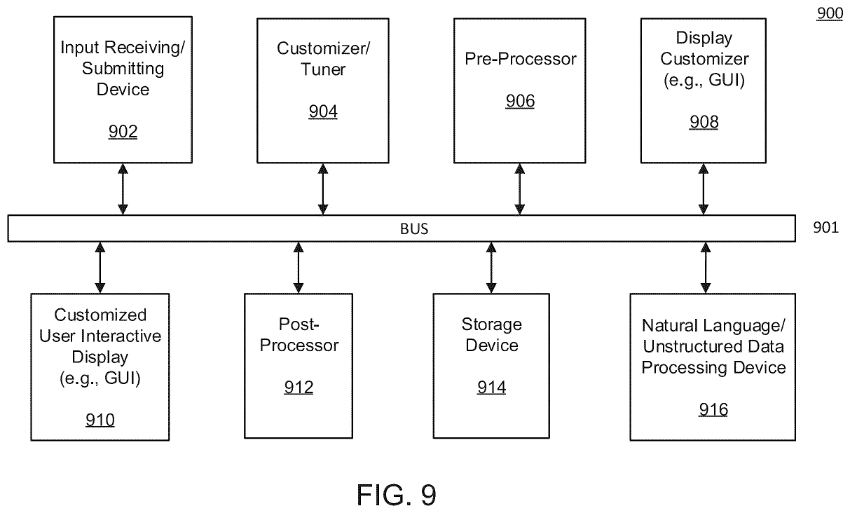

Moreover, it is to be appreciated that systems 200 and 900 described below with respect to FIGS. 2 and 9, are systems for implementing respective embodiments of the present invention. Part or all of processing system 100 may be implemented in one or more of the elements of systems 200 and 900.

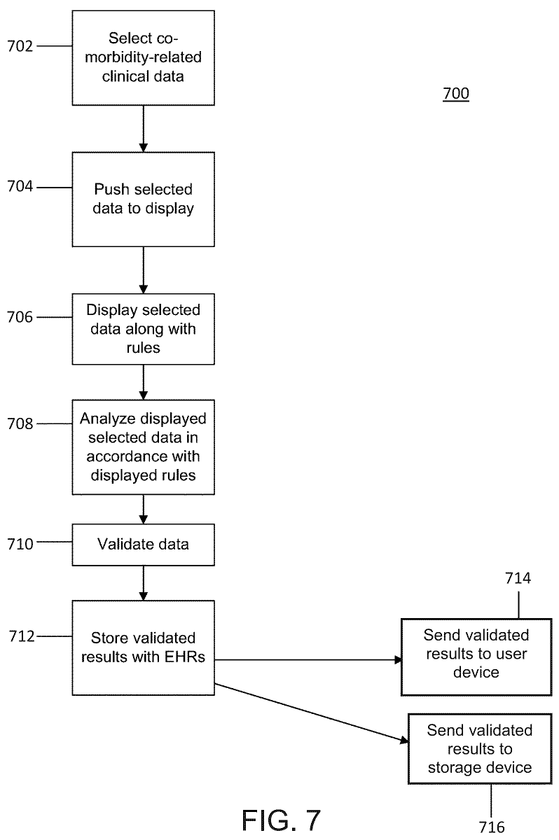

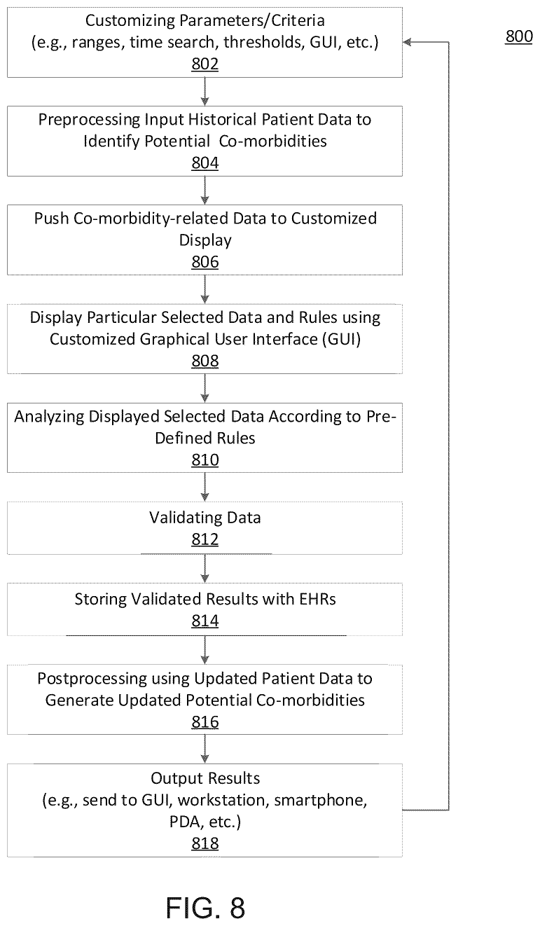

Further, it is to be appreciated that processing system 100 may perform at least part of the method described herein including, for example, at least part of methods 700 and 800 of FIGS. 7 and 8, respectively. Similarly, part or all of systems 200 and 900 may be used to perform at least part of methods 700 and 800 of FIGS. 7 and 8, respectively.

Referring now to FIG. 2, a block/flow diagram showing an exemplary high-level system architecture for automated determination, analysis, and/or presentation of patient co-morbidities 200 is illustratively depicted in accordance with one embodiment of the present principles.

The system 200 can include a database server 210 having memory in which patients' electronic health records (EHRs) 210 are stored, and an application server 212 having memory in which algorithms for identifying and documenting co-morbidities reside. Each server can include a processor, processing device and/or CPU, storage and memory. End users, such as emergency room clinicians, can interact with the system via a user interface (UI) 230 having a graphical interface or GUI. The results can be displayed by the system on the display of the UI 230 and stored in the EHR and/or stored in any appropriate storage medium 232 (e.g., server, PC, portable computing device, smartphone, etc.) in accordance with various embodiments of the present invention. The UI 230 can communicate with the database server 202, the application server 212, the EHR and/or storage medium 232 via a network 236. The network 236 can be a local area network (LAN), intranet, internet, or any other communication network as known to one skilled in the art.

Clinical data about a patient, represented in the patient's EHR 210, can come from several sources depending on the kinds of tests done on the patient-Laboratory (Lab) results 220, Unstructured (e.g., Radiology) 218, EKG data/reports 216, Echo 214, etc. Selected co-morbidities and/or comorbid conditions are sought which are high priority for the clinicians and/or physicians, and for hospital documentation. Not all clinical data is retrieved. Hence the inventive system displays selected co-morbidities and/or comorbid conditions known to effect severity of illness, treatment, outcome and ambulatory sensitive conditions.

Comorbid conditions are identified by algorithmic analysis of these data. These analyses are encoded as decision making rules. “Alert Fatigue” is avoided by selecting only relevant data, that is, data obtained through analysis in accordance with decision making rules and displaying and validating only these carefully selected results.

The Rule Processor component 224 executes these decision making rules. Execution of rule(s) associated with co-morbidity corresponds to determining, based on the patient's clinical data, if the co-morbidity holds. The Rule processor can keep physicians up-to-date with ICD-10 and other changes, such as sepsis definitions, so that accurate documentation of important variables is validated and recorded. With respect to the rules required for analysis, end-users of the inventive system can specify the kind of rules they want the system to use for analysis in an intuitive way. For example, one hospital can use RIFLE criteria (Risk, Injury, and Failure; and Loss, and End-stage kidney disease) for determining kidney failure and another can use KDIGO criteria (Kidney Disease: Improving Global Outcomes); the end-user can easily select the rule to be used.

Clinical data in EHR is heterogeneous, i.e., possesses varying formats. Often lab data/reports 220 are structured and are represented neatly in tabular form. On the other hand, Echo reports 214 and other unstructured input data 218 can include unstructured text and/or other types of data. Analysis of such text can be aided by Natural Language Processing technologies such as linguistic parsers, parts of speech taggers, entity extractors, etc., using the NLP Anaylzer component 222.

In various embodiments, a customizer 226 can be employed to adjust any of a plurality of features of the system, including, for example, adjusting the time-search parameters, customizing a GUI for a particular user/type of user/device/etc., adjusting ranges (e.g., laboratory ranges, hospital/physician preferences, etc.) in accordance with the present invention. A pre-processor and/or post-processor 228 can be employed to perform pre-and post-processing, respectively, on input patient data in accordance with various embodiments of the present invention.

The patient's co-morbidities identified by the algorithm can be presented, e.g., displayed, to the ER clinician via the GUI on the UI 230. The clinician simply needs to validate the displayed co-morbidities. To assist in the validation, the display may also include the rule(s) corresponding to the co-morbidity as well as the associated patient clinical data. For instance, if anemia is identified as a co-morbidity, the GUI 230 will present current hemoglobin/hematocrit values, and past values, if they are available. The GUI 230 will also show the rules for determining anemia based on hemoglobin/hematocrit values. Displaying present and past values quickly assists the clinician in determining whether the anemia is acute or chronic The ER clinician makes the final decision about including or excluding the co-morbidities presented by the algorithm. All of the selected co-morbidities become a part of the patient's EHR.

A use scenario to illustrate the novel invention is presented. A patient comes into the ER complaining of chest pain. The Attending Physician (AP) orders a series of tests. The cardiac tests (EKG and cardiac enzymes) reveal no underlying problems. But the lab tests have identified a couple of other unrelated issues-low potassium and high creatinine. The busy AP wants these findings to be reported to the patient's primary for follow up. The AP clicks on a link labeled co-morbidities on the ER's IT system. The two abnormal lab findings are displayed. AP clicks on a checkbox validating that these are indeed abnormal and clicks “ok” and the co-morbidities are stored as part of the patient's EHR. The patient is discharged and counseled to see his primary care physician, e.g., “Primary”. In this use scenario, the Primary is a participant in the Regional Health Network and thus has access to his patient's EHR. Upon the patient's discharge from the ER, an email is automatically sent alerting the Primary of the patient's visit to the ER. The email contains a link to the patient's EHR. The patient makes an appointment to follow up with the Primary. When the patient meets the Primary, the Primary opens the email from the ER and clicks on the patient's EHR link. The link to the co-morbidities is displayed prominently in the EHR. On clicking it, the Primary sees a very succinct summary of the co-morbidities and begins to discuss them with the patient.

In this use scenario, the AP responded to the low potassium finding by instructing the nurse to administer supplemental potassium to the patient. In other words, the patient was treated for this condition in the ER. For reimbursement purposes, the treated co-morbidity must be documented and named (e.g. hypopotassemia) which, as the use scenario indicates, is intrinsic to the novel system.

In current ER practices, the clinician has to explicitly search the EHR results and reports for significant clinical indicators of a comorbid condition and then document them in the record, which is a time consuming process that is prone to documentation errors and omissions especially in busy ERs. In contrast, the inventive system described herein automatically pushes the co-morbidities to the ER clinician and presents them succinctly in appropriate narrative form. All that the clinician has to do is simply validate them.

The novel system is more sophisticated than straightforward Clinical Decision Support (CDC) systems in that the present system provides information to the physician, and displays the information along with the rule that allows the physician to analyze and verify the displayed data to determine whether or not a comorbid condition is present. Upon verification, the information is documented in the patient's EHR. The inventive system can search multiple fields (e.g., clinical data), within the electronic record with one click (e.g., mouse click, on-screen button push, etc.), and then can present the results to a user (e.g., physician, nurse, administrator, etc.) in real-time (e.g., effectively instantaneously). For certain co-morbidities, such as anemia or kidney injury, the inventive system also searches past records (prior history) and presents previous and present values to the physicians to help determine whether the condition is chronic or acute.

In one embodiment, algorithms (and supporting computing infrastructure) can identify over two dozen co-morbidities from Lab results, vital signs and body weight and height. The algorithms and associated computing infrastructure can be integrated into an Information Technology (IT) system, such as, for example, the Cerner IT system at Stony Brook University Medical Center's ER. In the Cerner system, the clinician interacts with ER patient's data via a UI 230 which is a browser-centered dashboard. However, it is noted that the present invention can be integrated and/or employed with any IT system in accordance with various embodiments.

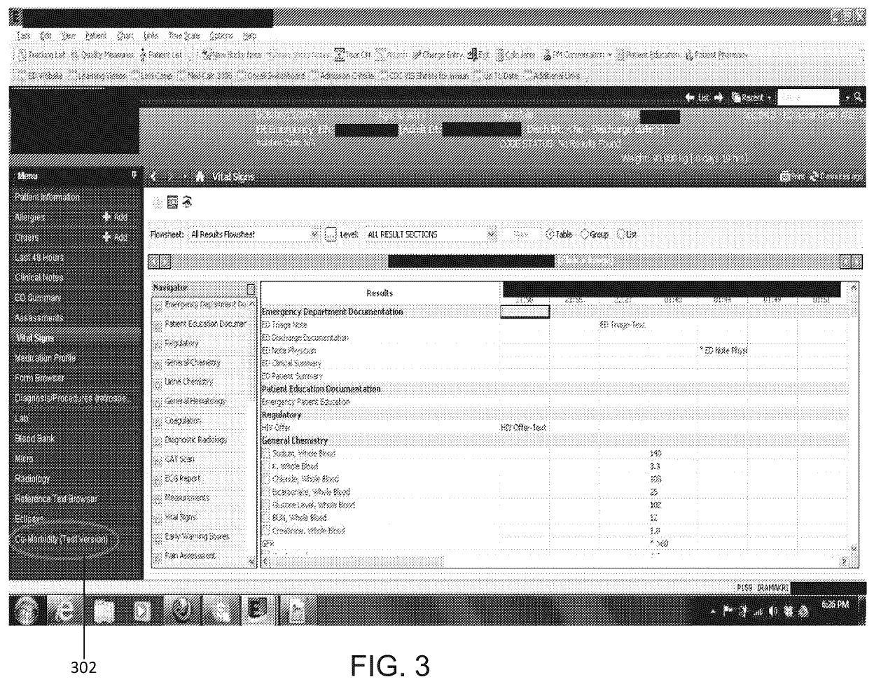

Referring now to FIG. 3, a diagram showing an exemplary display of a clinician's view of a patient's information is illustratively depicted in accordance with an embodiment of the present invention. This exemplary screen shot is a fragmentary snapshot of the clinician's view of a patient's information on this dashboard or UI. To this dashboard a link labeled “co-morbidity” is added; this link is enclosed within the oval 302 in FIG. 3. Upon clicking this link, the co-morbidity application residing on the application server 212 is invoked. The application fetches the patient's EHR from the EHR database 210 system for analysis.

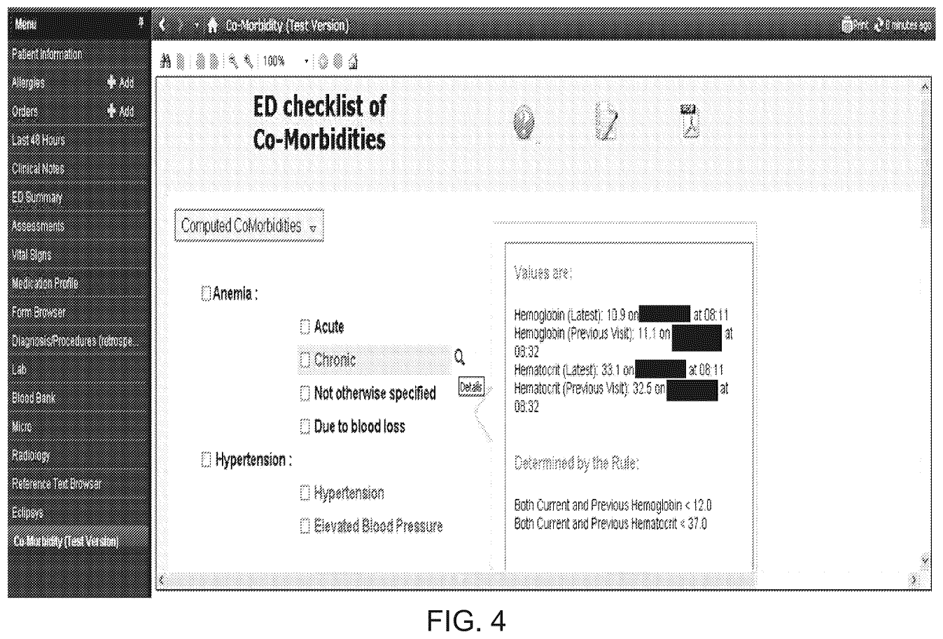

Referring now to FIG. 4, an exemplary display of an emergency room diagnosis (ED) checklist of co-morbidity is illustratively depicted in accordance with an embodiment of the present invention. Upon completion of the analysis, the applicable co-morbidities, e.g., “co-morbidity-related clinical data”, can be displayed in the ED checklist of Co-Morbidities, which in the exemplary embodiment shown in FIG. 4 shows two co-morbidities: anemia and hypertension. In one embodiment, hovering over co-morbidity displays the associated lab value, the normal ranges and the corresponding rule morbidity, such as anemia—chronic, as shown in FIG. 4. As illustrated, the values for chronic anemia include the latest hemoglobin, a previous hemoglobin, the latest hematocrit and a previous hematocrit, all values including the date and time the value was measured. Also illustrated is the determination by the rule “Both Current and Previous Hemoglobin <12.0; Both Current and Previous Hematocrit <37.0”. Hence, according to the rule, this patient has chronic anemia. The clinician reviews these values and clicks on the “square box” adjacent to the co-morbidity, e.g., chronic, if the determination made and displayed by the system is valid.

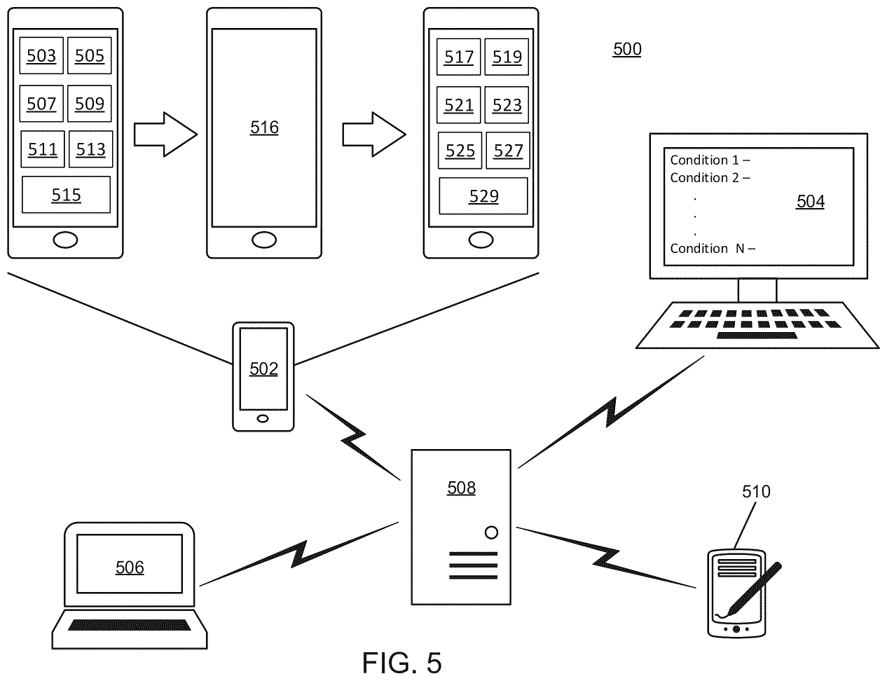

Referring now to FIG. 5, a diagram showing an exemplary high-level system architecture 500 for automated determination, analysis, and/or presentation of patient co-morbidities is illustratively depicted in accordance with an embodiment of the present invention. In an exemplary embodiment of the present invention, the system 500 can automatically determine, analyze, and present patient co-morbidities in a customized, personalized layout, which can be adjusted to particular user needs and/or customized for optimal use efficiency for any of a plurality of device displays (e.g., smartphone 502, PC 504, laptop 506, PDA, etc.), which can receive data from, for example, a server 508 (e.g., database server, application server, etc.). There are a plurality of different, customizable display (e.g., GUI) formats that can be employed in accordance with various aspects of the present invention.

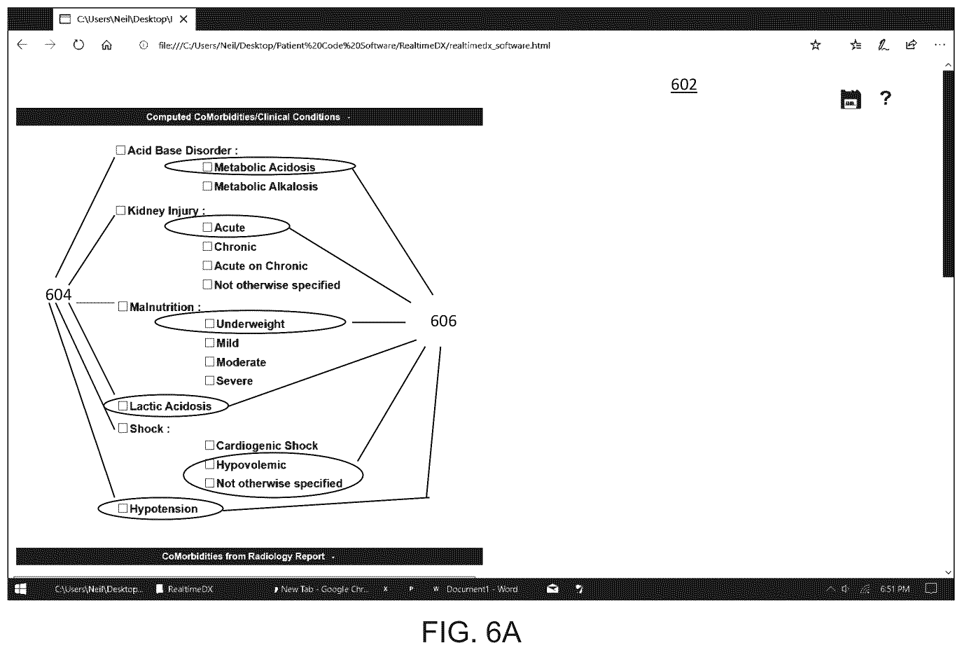

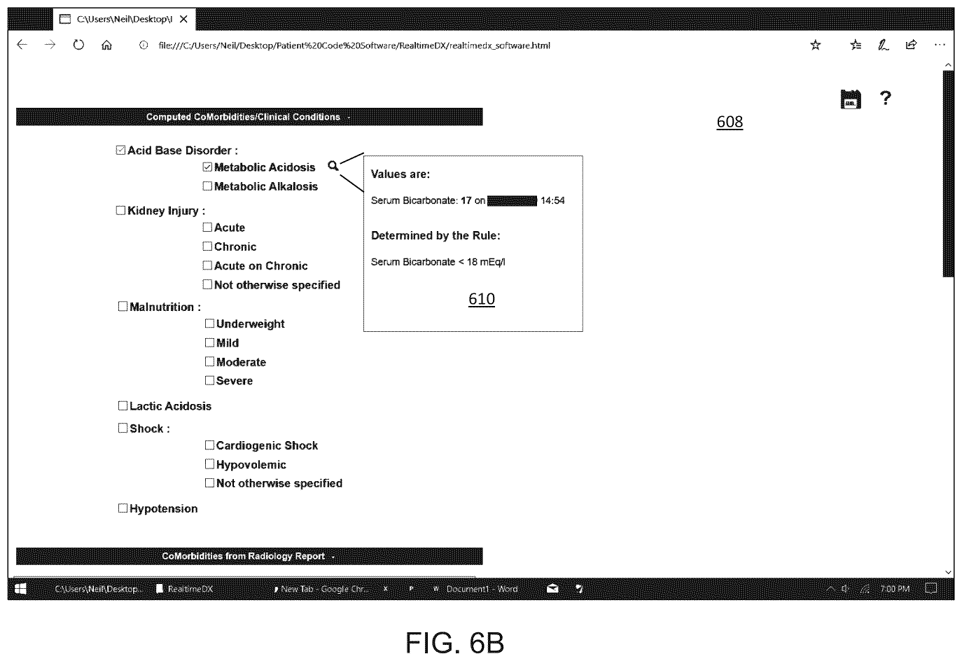

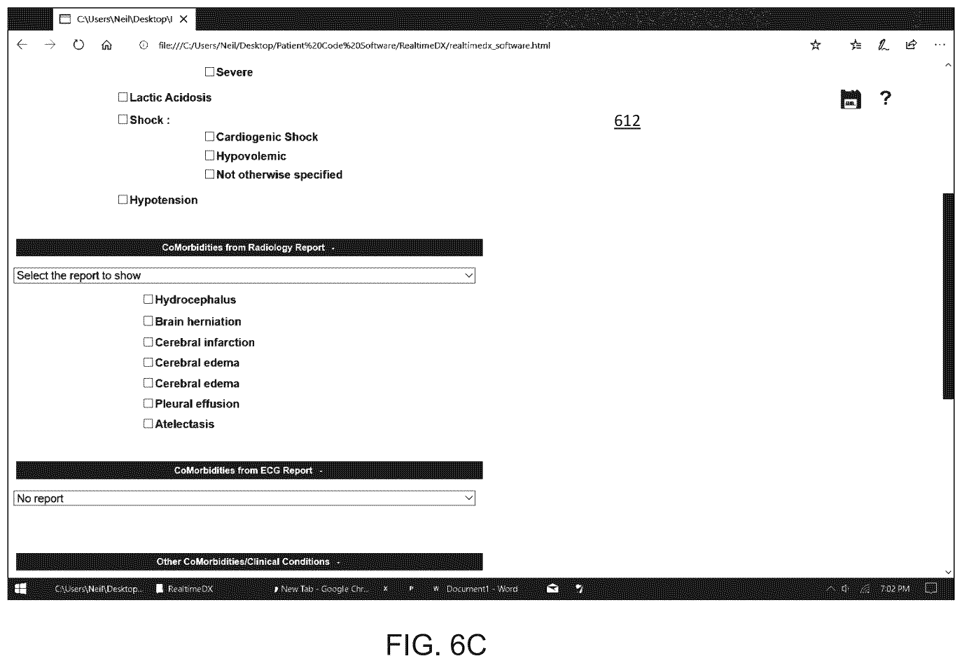

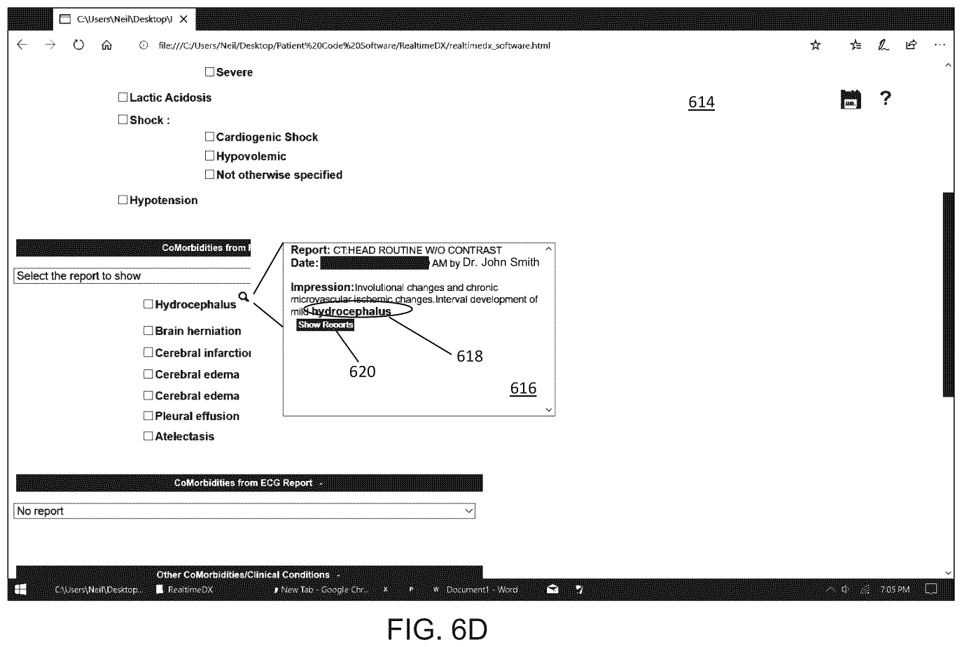

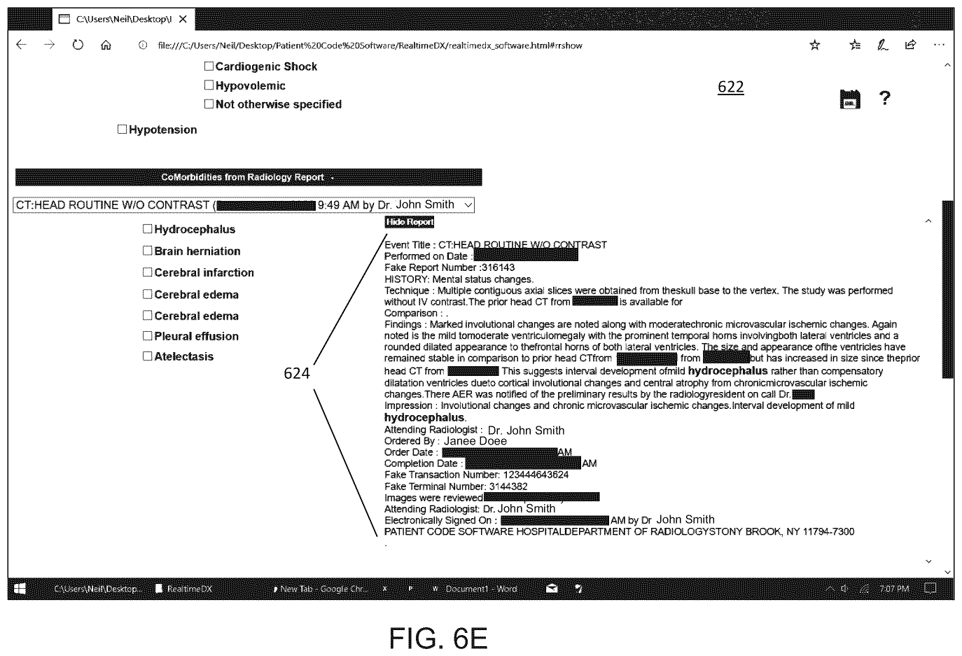

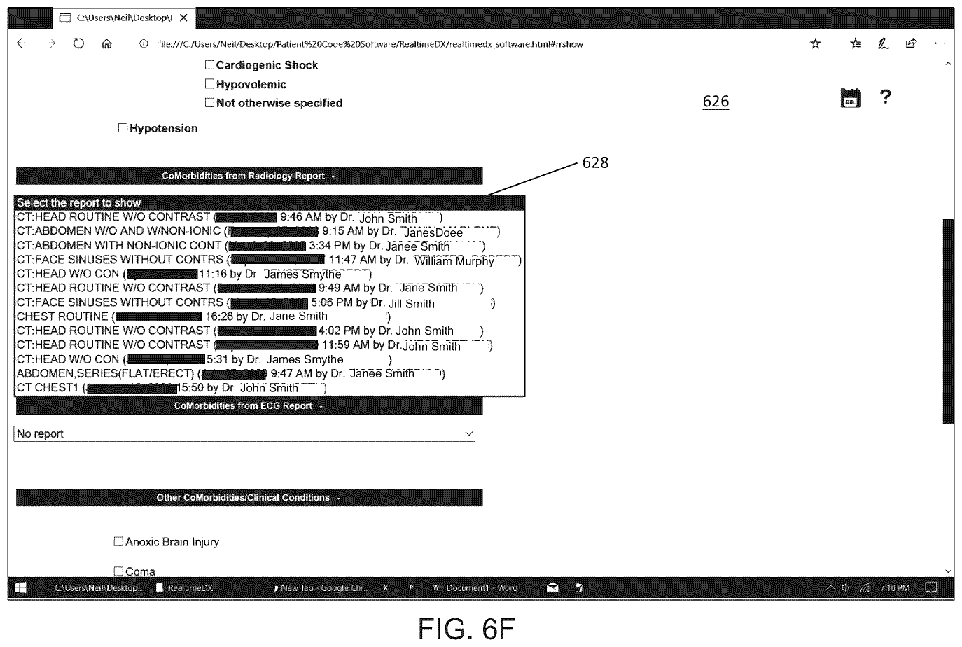

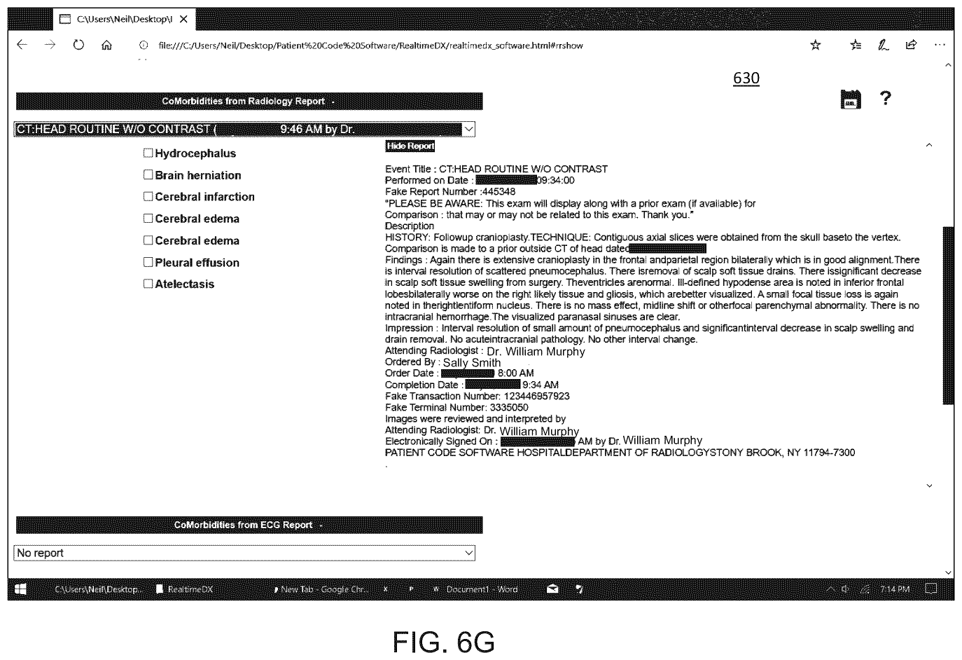

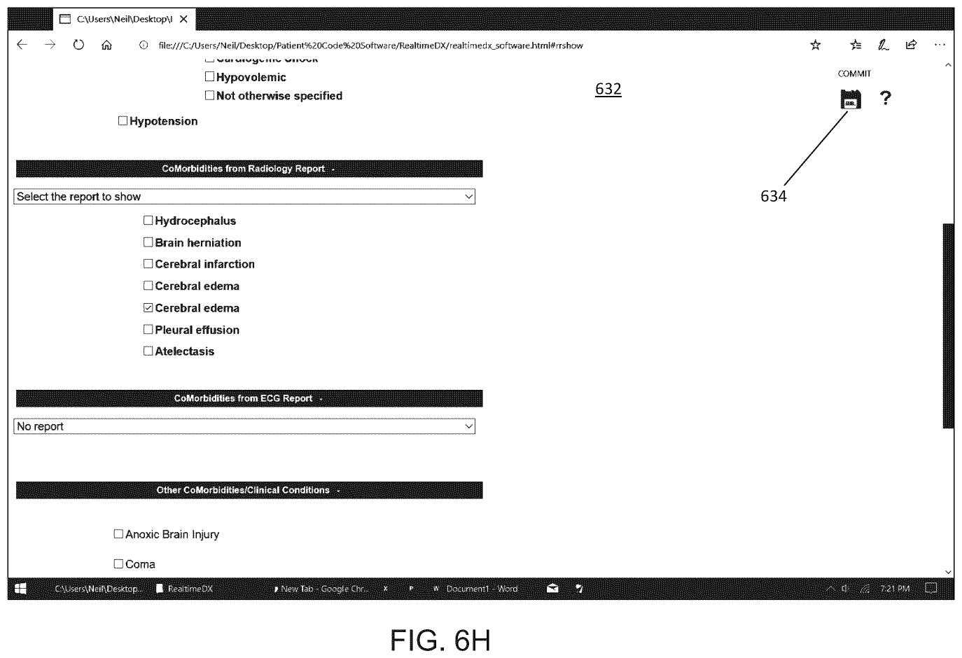

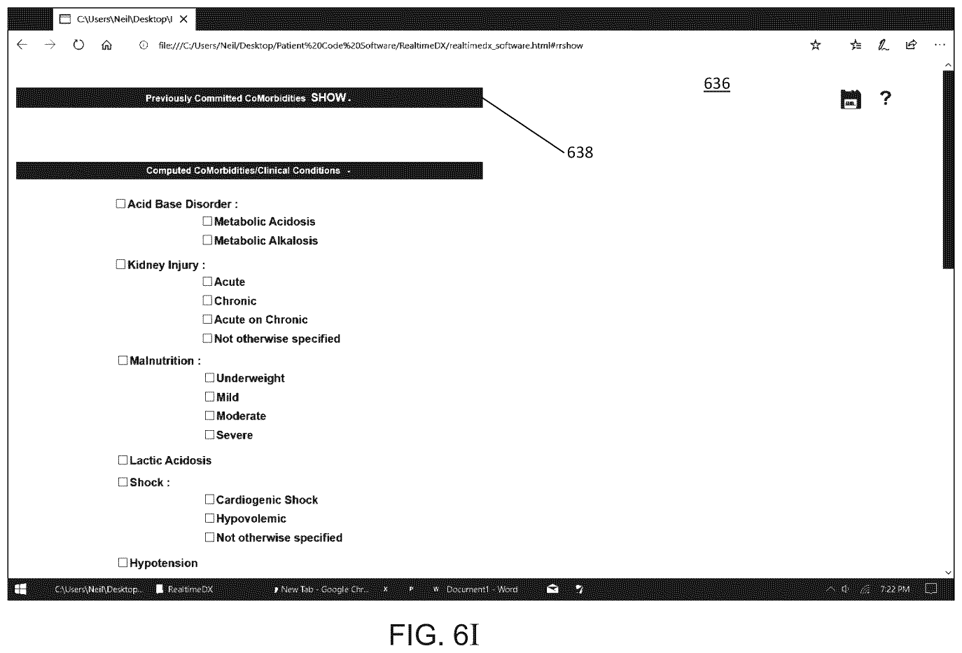

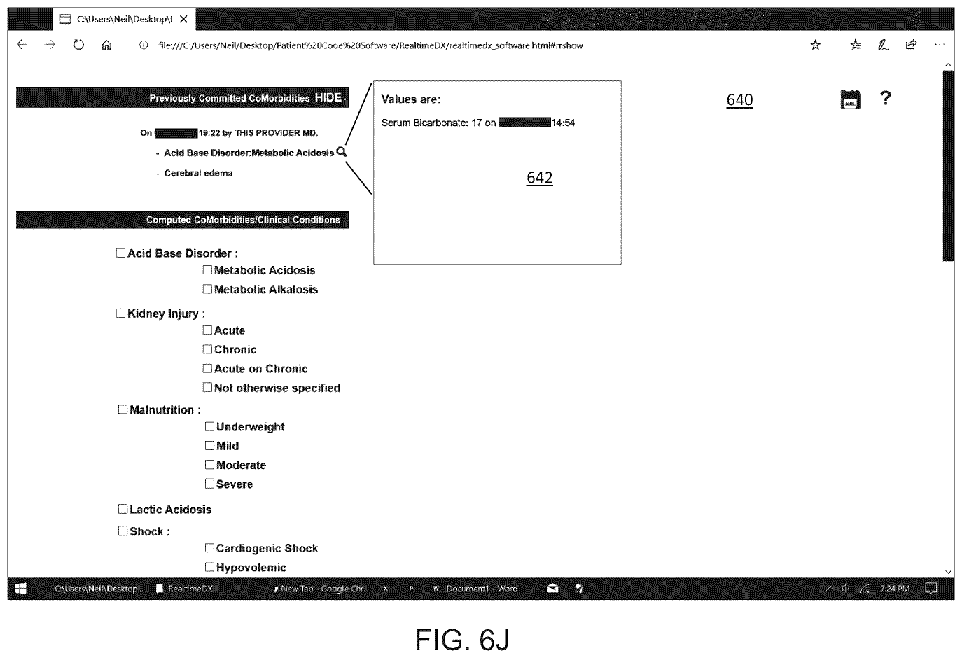

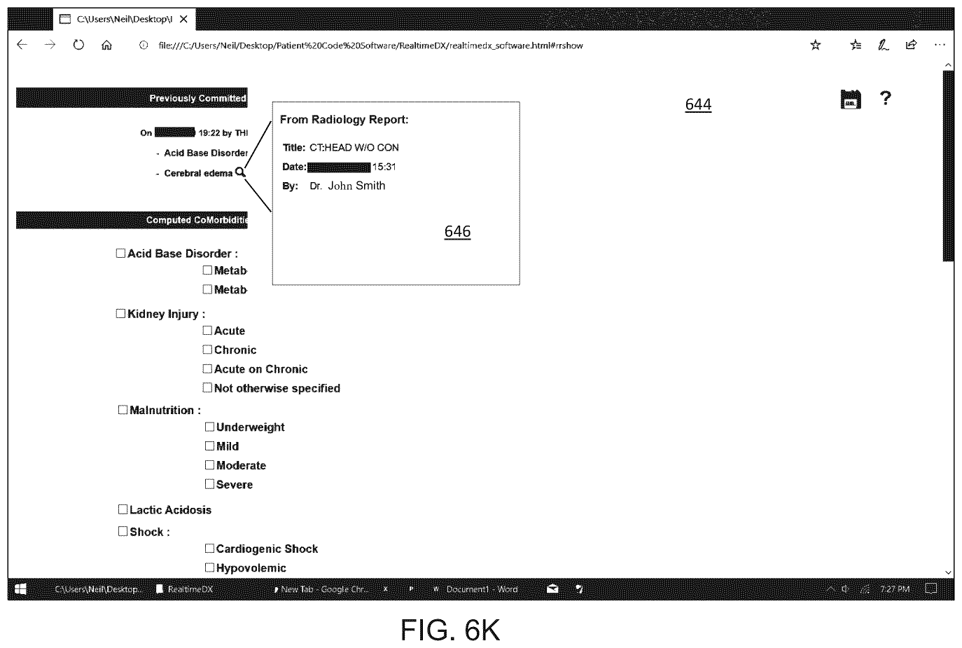

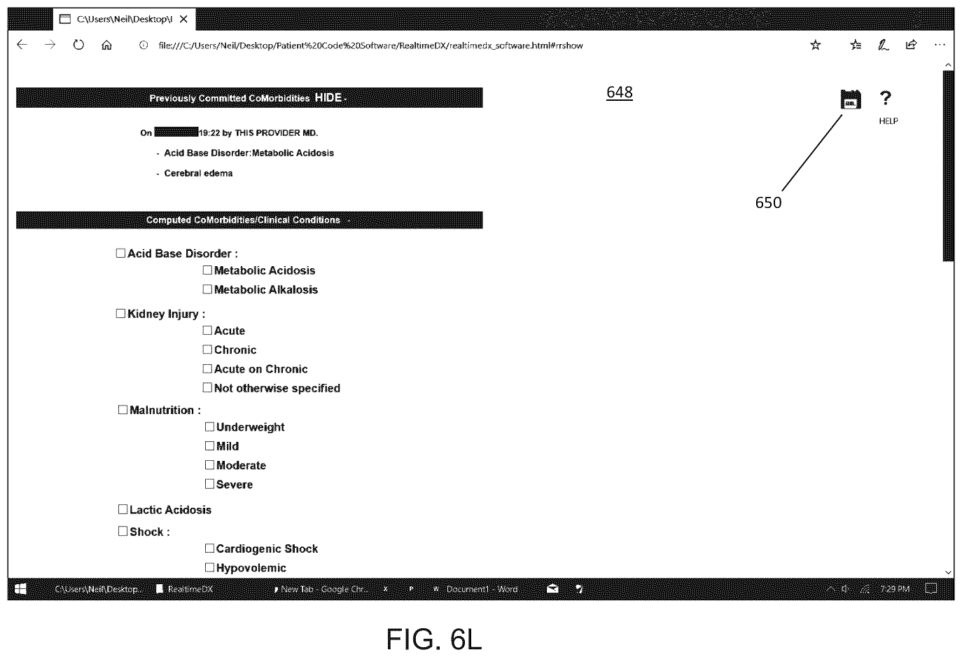

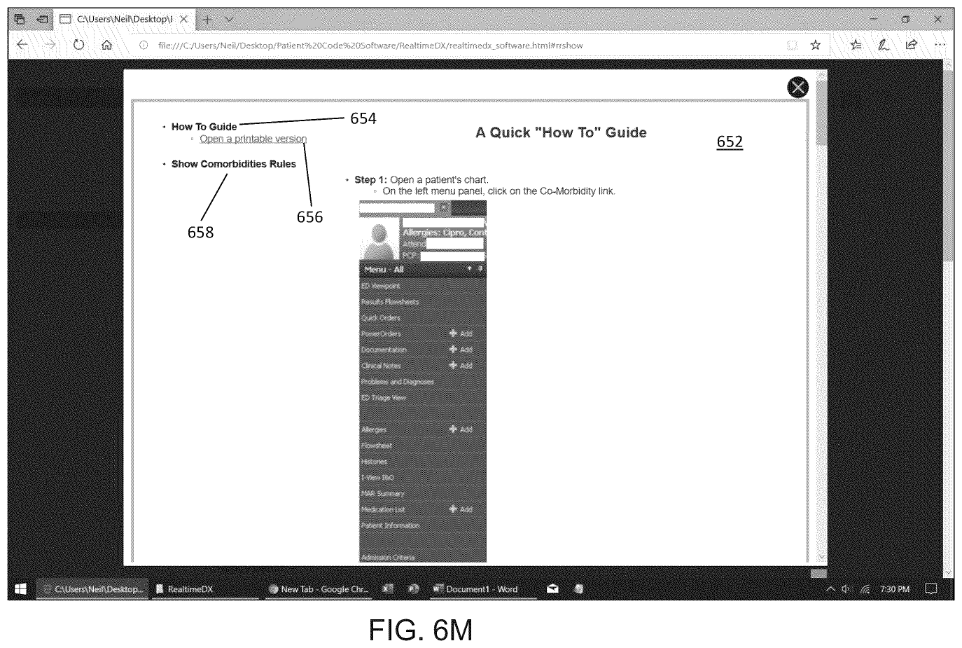

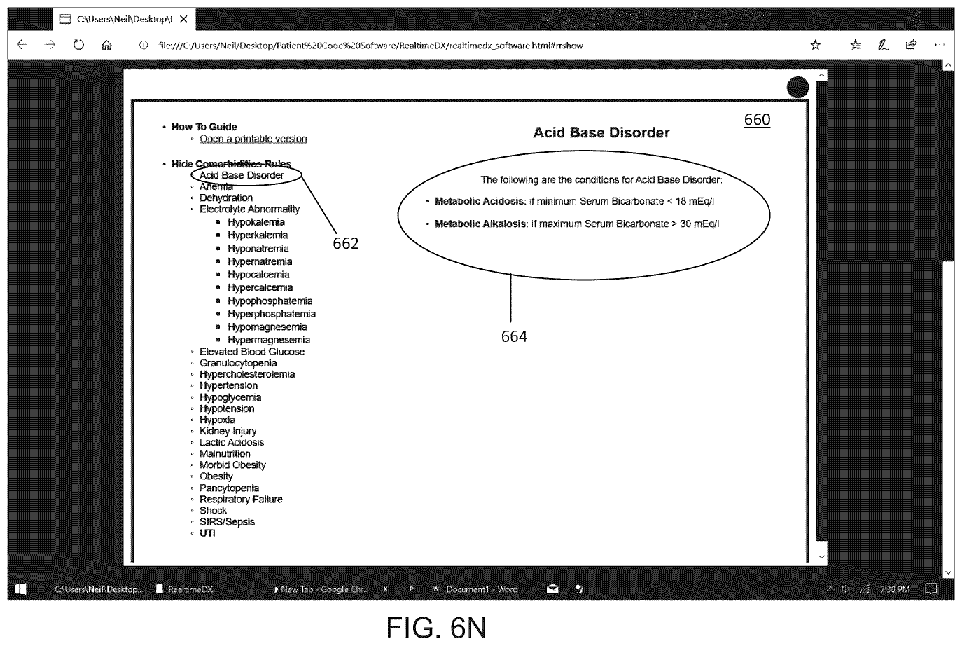

For example, a portable communication device 502 (e.g., cellular telephone, smartphone device, etc.) is known to have a relatively small screen, which can cause reading a list of options/conditions to be cumbersome and difficult to read. In an aspect of the present invention, a GUI can be generated and/or populated with options/conditions presented in a manner which increases usability and/or readability of the questions. In an exemplary embodiment of the present invention, the display screen of the portable communication device 502 can include one or more GUI buttons 503, 505, 507, 509, 511, and 513 corresponding to, for example, different computed co-morbidities/clinical conditions, similarly to the conditions shown hereinbelow with reference to FIG. 6A. The buttons 503, 505, 507, 509, 511, and 513 shown are exemplary, and it is to be appreciated that the buttons 503, 505, 507, 509, 511, and 513 can represent any sort of data types, and can include any number of buttons, configurations, and/or labels on the buttons 503, 505, 507, 509, 511, and 513. The buttons 503, 505, 507, 509, 511, and 513 can be customized, visually or functionally, to suit the needs of a particular user, device type, conditions, etc. in accordance with various embodiments of the present invention.

In various embodiments of the present invention, a long press/long tap (e.g., employed on touchscreens, smartphones, tablets, smartwatches, etc. to the long press or long tap increases the flexibility of the user interface. The “short press” or “short tap” (releasing right away) performs one operation, while pressing/tapping and holding that same button for a short time activates another) of any of the buttons 503, 505, 507, 509, 511, and 513 can bring up further details regarding the pressed button, which can be displayed as a pop-up window 515 (e.g., either being displayed in front of other buttons 503, 505, 507, 509, 511, and 513 or next to the buttons), or on a new display screen 516 (e.g., to display further details regarding any of the clicked buttons).

In some embodiments, additional customizable GUI interface displays can be utilized, and can include buttons 517, 519, 521, 523, 525, and 527 corresponding to, for example, co-morbidities from a radiology report, and can further include a pop-up window 529 (e.g., either being displayed in front of other buttons 517, 519, 521, 523, 525, and 527 or next to the buttons), or in a new display screen (not shown), similarly to the display screen 516 (e.g., to display further details regarding any of the clicked buttons). It is to be appreciated that although the buttons 517, 519, 521, 523, 525, and 527 are shown in a particular layout, and are described as representing particular data from a particular report (e.g., co-morbidities from a radiology report), the buttons 503, 505, 507, 509, 511, 513, 517, 519, 521, 523, 525, and 527 can be customized to represent any sort of data in any sort of customized layout in accordance with various aspects of the present invention.

In some embodiments, similarly to the customized GUI described above with reference to the portable communication device 502 (e.g., cellular telephone, smartphone, etc.), a personal computer (PC) and/or virtual machine (VM) 504, a laptop computer 506, and/or any type of portable computing device 510 (e.g., personal digital assistant (PDA), tablet, phablet, etc.) can include a similar customizable interface/layout for interacting with the system 500. In some aspects of the present invention, rather than presenting the options/conditions as shown with reference to the personal communication device 502, the interface can display the options/conditions as an ordered list (e.g., on a single or multiple screens), in separate smaller windows on a computer screen, and/or highlight highly prioritized options/conditions to improve speed and efficiency of presenting and/or answering interview questions for one or more users. For example, the exemplary display screen of the PC 504 can include customizable lists, which can emphasize (e.g., highlight, bold, underline, etc.) particular options/conditions in accordance with various embodiments of the present invention, which will be described in further detail hereinbelow with reference to FIGS. 6A-6N.

In accordance with aspects of the present invention, the GUIs shown in, for example, 502, 504, 506, and 508 can be customized to quickly and efficiently display the most relevant data for a particular user, task, condition, etc., and the data can be presented to users (e.g., health care workers) in real-time to aid in, for example, physician decision making regarding particular treatment for particular patients. Thus, the customizable GUIs of the system 500 can reduce the time spent by users (e.g., health care workers) in completing tasks, and increasing the accuracy and efficiency of determination, analysis, and presentation of patient co-morbidities in accordance with various embodiments of the present invention.

Referring now to FIGS. 6A-6N, a plurality of exemplary customized graphical user interface (GUI) display/interaction screen shots are illustratively depicted in accordance with embodiments of the present invention. In FIG. 6A, the screen 602 displays computed diagnoses and/or co-morbidities based on, for example, lab results and vitals for a patient run against an algorithm (e.g., Rules Engine). The left-most list 604 represents diagnosis groups, and the sub-groups listed are the different types for a particular diagnosis, and can be highlighted/recolored 606 to show suggested diagnosis based on the algorithmic results in accordance with various embodiments of the present invention. In FIG. 6B, the screen 608 shows that hovering over the terms can display the values 610 that accounted for that diagnosis being displayed, as well as an algorithm from the Rules Engine for that term.

In FIG. 6C, the screen 612 shows an exemplary radiology report section based on what may have been found in radiology reports for the patient. This can utilize Natural Language Processing in the Rules Engine to process the reports in accordance with embodiments of the present invention. The ECG Report section (and other types of report sections) also functions the same way but with different diagnoses, and thus different GUI features in some embodiments of the present invention. In some embodiments, as shown in the screen 614 in FIG. 6D, hovering over terms will bring up the area(s) in the Impression section 616 of the report where the highlighted keyword(s) 618 (based on the Rules Engine) were found for that diagnosis. The “Show Reports” button 620 can bring up the entire report on the right-side.

In FIG. 6E, the screen 622 shows details of a report in block 624, and the details can include highlighted portions to emphasize particular portions of the report in accordance with embodiments of the present invention. In FIG. 6F, the screen 626 includes a button for clicking on the “Select the report to show” drop-down 628, which can bring up a list of all the radiology reports found for the patient, whether or not a diagnosis was found in the report. Selecting a report can display the entire report on the right-side of the screen, as shown in screen 630 of FIG. 6G, and there will be no highlighted keywords if no diagnoses were found in accordance with embodiments of the present invention.

In FIG. 6H, after the provider/clinician has validated the diagnoses, they can click on the appropriate checkboxes on the screen 632 and click on the “COMMIT” button 634 on the top-right (diskette icon), which can create a “Previously Committed CoMorbidities” section 638 at the top of the screen, as shown in screen 636 of FIG. 6I in accordance with embodiments of the present invention.

In FIG. 6J, a screen 640, including a “SHOW” button, can be included, and clicking on the “SHOW” can expand this section and display the previously committed diagnoses, as shown in block 642. The date and time, the provider's name, and the diagnoses they selected can be displayed in block 646, as shown in screen 644 of FIG. 6K. Hovering over a diagnosis can display the values at the time that was committed, as shown in block 642 of FIG. 6J. If the diagnosis is from a Radiology or ECG report, then it can display the info from the specific report where that diagnosis was found in accordance with embodiments of the present invention.

In FIG. 6L, a screen 648 can include several “clickable” icons in accordance with embodiments of the present invention. For example, clicking on the “?” icon 650 on the top-right can bring up the HELP menu. In FIG. 6M, clicking on the “How To Guide” 654 on screen 652 can bring it up on the right-side. Clicking on the “Open a printable version” 656 can bring up a MS Word doc version. Clicking on the “Show Comorbidities Rules” 658 can bring up the list of diagnoses under it. In FIG. 6N, clicking on a diagnosis 662 can bring up the algorithm 664 (e.g., rules) on the right-side of the screen 660 in accordance with embodiments of the present invention. It is to be appreciated that other customizable screens/buttons/etc. can be created and/or utilized in accordance with various embodiments of the present invention, and the presented screen shots are intended to be merely illustrative of some embodiments of the present invention, and are not intended to be limiting to these specific embodiments and/or screen layouts.

Referring now to FIG. 7, a block/flow diagram showing a method 700 for automated identification, documentation, and displaying of co-morbidities from patient Electronic Health Records (EHRs) is illustratively depicted in accordance with various embodiments of the present invention. In block 702, co-morbidity-related clinical data can be selected in accordance with one or more rules. Clinical data can include data from laboratory tests, Radiology, EKG, Echo reports, etc. The data is selected based on analysis using Rule processor 224 and/or the Pre-and/or Post processors 228. In block 704, the selected clinical data is pushed to a display. In block 706, the selected clinical data is displayed, (e.g., via a customized GUI 230), to the ER physician, along with the one or more rules.

In some embodiments, in block 708, a physician analyzes the displayed selected clinical data in accordance with the displayed rules. In block 710, acknowledgement or validation of the results can be obtained from the ER physician. In block 712, validated results are stored in the EHR. In block 714, validated results can be sent to any of a plurality of types of user devices (e.g., PC, laptop, smartphone, tablet, etc.) for viewing the results. In some embodiments, the user devices can include customized graphical user interfaces (GUIs) with personalized interactive displays, which will be described in further detail hereinbelow. In block 716, validated results can be stored in any of a plurality of storage devices (e.g., server, PC hard drive, persistent storage devices, etc.) for later access and/or analysis, in accordance with various embodiments of the present invention.

In some embodiments, clinical data is obtained in the ER but other hospital and/or care center data can provide data, and the obtained data is processed, that is, the data is placed in a format for analysis. In some embodiments, NPL Analyzer 222 can be used for formatting and/or processing the data.

Referring now to FIG. 8, a block/flow diagram showing a method 800 for automated determination, analysis, and/or presentation of patient co-morbidities is illustratively depicted in accordance with an embodiment of the present invention. In block 802, customization of parameters and criteria (e.g., laboratory ranges, user/physician selected ranges, time search adjustments, threshold definition, GUI customization, etc.) can be performed in accordance with various embodiments of the present invention.

In block 804, potential co-morbidities can be identified and/or determined by preprocessing received input (e.g., historical patient data/records). In block 806, co-morbidity related data can be pushed (e.g., formatted and sent) to a customized GUI, and in block 808, particular selected data and/or rules can be displayed on the customized GUI in accordance with embodiments of the present invention. The displayed selected data can be analyzed according to pre-defined rules in block 810, and the data can be validated in block 812 in accordance with embodiments of the present invention.

Exemplary rules for co-morbidity analysis can include, but are not limited to the following:
Anemia
   Acute
      Hemoglobin/Hematocrit<low AND previous visit
      Hemoglobin/Hematocrit>low
   Chronic
      Hemoglobin/Hematocrit<low AND previous visit
      Hemoglobin/Hematocrit<low
   Not otherwise specified
      Hemoglobin/Hematocrit<low AND no previous visit
      Hemoglobin/Hematocrit
   Due to blood loss
Pancytopenia
   Due to Chemotherapy.
      White Blood Cell (WBC) Count<low, Red Blood Cell (RBC) Count<low, and Platelet (PLT) Count<low
   Due to Drugs.
      WBC Count<low, RBC Count<low, and PLT Count<low
   NOS
      WBC Count<low, RBC Count<low, and PLT Count<low
Acid Base Disorder
   Metabolic Acidosis.
      Serum Bicarbonate<18 mEq/l
   Metabolic Alkalosis
      Serum Bicarbonate >30 mEq/l
Kidney Injury
   Acute
      Latest Creatinine>1.2 and Previous Creatinine<=1.2 OR Latest Creatinine=1.5×Previous Creatinine
   Chronic
      Previous and Latest Creatinine both >1.2
      Stage 1.
         Glomerular Filtration Rate (GFR)>=90
      Stage 2
         GFR>=60 and <90
      Stage 3
         GFR>=30 and <60
      Stage 4
         GFR>=15 and<30
      Stage 5
         GFR<15
   Acute on Chronic
      Previous and Latest Creatinine both >1.2 AND Latest Creatinine=1.5×Previous Creatinine
      Stage 1
         GFR>=90
      Stage 2
         GFR >=60 and<90
      Stage 3.
         GFR>=30 and <60
      Stage 4
         GFR>=15 and <30
      Stage 5
         GFR<15
   Not otherwise specified
      Latest Creatinine>1.2 AND Previous Creatinine is not available
Malnutrition
   Underweight
      Body Mass Index (BMI)>=18.0 and <20.0
   Mild.
      BMI>=17.0 and<18.0
   Moderate.
      BMI>=16.0 and<17.0
   Severe
      BMI<16.0
Morbid Obesity
   BMI>40
Dehydration
   The ratio of BUN/creatinine>20 for age up to 1 year OR The ratio of Blood Urea Nitrogen (BUN)/creatinine>30 for age over 1 year
Elevated Blood Glucose
   Hyperglycemia
      Glucose Value>140
   Diabetes Type 1 with Hyperglycemia
      Glucose Value>140
   Diabetes Type 2 with Hyperglycemia
      Glucose Value>140
   Diabetes with Hyperglycemia and Nephropathy
      Glucose Value>140 AND Creatinine Value>1.2
Hypoxia
   Pulse Oximetry level<90
Granulocytopenia
   Neutrophil level<low
Lactic Acidosis
   Lactic Acid level>2.0
Electrolyte Abnormality
   Hypokalemia
      Potassium<low
   Hyperkalemia
      Potassium>high
   Hyponatremia
      Sodium level<low
   Hypernatremia
      Sodium level >high
   Hypocalcemia
      Calcium level<low
   Hypercalcemia
      Calcium level>high
   Hypophosphatemia
      Phosphorus level<low
   Hyperphosphatemia
      Phosphorus level>high
   Hypomagnesemia
      Magnesium level<low
   Hypermagnesemia
      Magnesium level>high
Obesity
   BMI>30 and <40
Hypertension
   Hypertension
      Systolic Blood Pressure (BP)>140 or Diastolic BP>90
   Elevated Blood Pressure
      Systolic BP>140 or Diastolic BP>90
Urinary Tract Infection (UTI)
   Urinalysis, WBC Count >5 AND either Urinalysis, Leukocyte Esterase is NOT NEGATIVE or Urinalysis, Nitrite is NOT NEGATIVE
   Catheter Associated
Systemic Inflammatory Response Syndrome (SIRS)
   SIRS due to noninfectious process
      On current encounter, 2 or more of the following are present:
         Temperature<36.0 or >=38.3
         Heart Rate (HR) >90
         Respiratory Rate (RR)>20
         Bands>10%
         Serum WBC<4,000 or >12,000
         ***noninfectious causes such as trauma, burn, pancreatitis, other pro-inflammatory conditions.
   Sepsis due to suspected or actual infection
      On current encounter, 2 or more of the following are present:
         Temperature<36.0 or >=38.3
         HR>90
         RR>20
         Bands>10%
         Serum WBC<4,000 or >12,000
         **due to suspected or actual infection and values not easily explained by co-existing conditions
   Severe Sepsis
      If any of the following exists:
         Lactic Acid>2.0
         Systolic BP<90
         MAP<65
         Total Bilirubin>2
         Creatinine>=2.0
         PLT Count<100k
         Prothrombin Time, INR>1.5
         Activated Partial Thromboplastin Time (aPTT)>60 seconds
         ***in absence of other causes of organ dysfunction
   Septic Shock
      If any of the following exists:
         Lactic Acid>=4.0
         Systolic BP<90 (unresponsive to fluid bolus)
         MAP<65 (unresponsive to fluid bolus)
Respiratory Failure
   Acute
      PO2<=60 or Pulse Oximetry<=90
   Chronic
      Current PCO2 or Current ETCO2>45 AND Previous PCO2 or Previous ETCO2>45
   Acute on Chronic
      PO2<=60 or Pulse Oximetry<=90 AND Current PCO2 or Current ETCO2>45 AND Previous PCO2 or Previous ETCO2>45
Shock
   Cardiogenic Shock
      Systolic BP<90 AND Cardiac Troponin>normal high
   Hypovolemic
      Systolic BP<90
   Not otherwise specified.
      Systolic BP<90
Hypotension
   Systolic BP<90
   ***Responsive to fluid resuscitation
Hypoglycemia
   Diabetes Type 1 with Hypoglycemia
      Glucose level<low
   Diabetes Type 2 with Hypoglycemia
      Glucose level<low
HyperCholesterolemia
   Total Cholesterol level>200

### Radiology Comorbidities

Pleural effusion
   Search for “effusion”
      Exudative
      Transudative
      Inflammatory effusion
      Malignant effusion
Pneumonia
   Search for “Pneumonia”
      Aspiration
      Bacterial
      Viral
      Suspected gram negative
Bronchiectasis
   Search for “Bronchiectasis”
      with acute exacerbation
      with lower respiratory infection
      with pneumonia
Acute Bronchitis
   Search for “bronchitis”
obstructive airway disease
   Search for “obstructive”
      Chronic
      with acute lower respiratory infection
      with acute exacerbation
Pulmonary edema
   Search for “edema”
      Acute
      Chronic
Acute Respiratory Distress Syndrome (ARDS)
   Search for “ARDS”
Atelectasis
   Search for Atelectasis
Cerebral compression
   Search for “Mass Effect”, Midline Shift”, “Compression”
Cerebral infarction
   Search for “Loss of grey matter”, “infarction”
Cerebral edema
   Search for “Vasogenic edema”, “edema”
      Cerebral edema with compression
Hydrocephalus
   Search for “Hydrocephalus”
      Normal Pressure Hydrocephalus
      Obstructive hydrocephalus
Subarachnoid hemorrhage
   Search for “Subarachnoid hemorrhage”
      Traumatic
      Non Traumatic
      Acute
      Chronic
      Acute on Chronic
Subdural Hematoma
   Search for “Subdural Hematoma”
      Traumatic
      Non Traumatic
      Acute
      Chronic
      Acute on Chronic
Epidural hematoma
   Search for “Epidural hematoma”
      Traumatic.
      Non Traumatic
      Acute
      Chronic
      Acute on Chronic
Cerebral hemorrhage
   Search for “Cerebral hemorrhage”
      Acute
      Chronic
      Acute on Chronic
Intraventricular hemorrhage
   Search for “Intraventricular hemorrhage”
      Acute
      Chronic
      Acute on Chronic
Cerebral Aneurysm
   Search for “Cerebral Aneurysm”
      Ruptured
      Non Ruptured
Brain Tumor
   Search for “Tumor”
      With compression
      With hemorrhage
Brain herniation.
   Search for “Herniation”
      with cerebral compression
      with cerebral edema
Sinusitis
   Search for “Sinusitis”
      Acute Sinusitis
      Chronic Sinusitis
      Acute on Chronic
Cerebral Abscess
   Search for “Abscess”
Cerebral Neoplasm
   Search for “Neoplasm”
      With compression.
      With hemorrhage
Pancreatitis
   Search for “Pancreatitis”
      Acute
         Alcohol induced
         Biliary
         Drug induced
         Not otherwise specified
      Chronic
         Alcohol induced
         Biliary
         Drug induced
         Not otherwise specified
Pancreatic cyst/pseudocyst
   Search for “Pancreatic cyst”
Intestinal Obstruction
   Search for “Obstruction”
      Intestinal obstruction with Ileus
      Intussusception
      Volvulus
Cholecystitis
   Search for “Cholecystitis”
      Acute
      Chronic
      Acute with Chronic
Gastritis
   Search for “Gastritis”
      Acute
         With bleeding.
      Chronic
         With bleeding
Peritonitis
   Search for “Peritonitis”
Cholecystitis
   Search for “Cholecystitis”
      Acute
      Chronic
      Acute with Chronic
Acute
   Search for “Enteritis”
      Due to C. diff
      Due to E. coli
      Bacterial

### ECG Comorbidities

Supraventricular tachycardia
   Search for “Supraventricular tachycardia”
Ventricular Tachycardia
   Search for “Ventricular Tachycardia”
Ventricular fibrillation
   Search for “Ventricular fibrillation”
Atrial fibrillation
   Search for “Atrial fibrillation”
      Paroxysmal
      Persistent
      Permanent
Myocardial infarction
   Search for “Infarction”, “Infarct”
      Old Myocardial Infarction
      Acute Myocardial Infarction
Atrial flutter
   Search for “Atrial flutter”
1st degree Atrioventricular (A-V) block
   Search for “1st degree A-V block”, “1st degree AV block”, “first degree A-V block”, “first degree AV block”

### Other Comorbidities

Anoxic Brain Injury
Coma
Seizure
Decubitis Ulcer
   Stage 1
   Stage 2
   Stage 3
   Stage 4
   Unstageable
   Unspecified Stage
Delirium
   Infection
   Drugs or toxin
   Metabolic Effect
   None of the Above
Encephalopathy
   Metabolic Encephalopathy
   Hepatic Encephalopathy
   Septic Encephalopathy
   Toxic Encephalopathy
   Hypertensive Encephalopathy
   Anoxic Encephalopathy
   Alcoholic Encephalopathy
   None
Other Non-Listed Comorbidities: {freetext}

It is noted that the above rules can be tuned (e.g., customized) to the requirements/preferences of individual users/hospitals/physicians, etc., in accordance with embodiments of the present invention. The present invention enables customization depending on, for example, variations in laboratory normal ranges as well as hospital and physician preference and or tolerance (e.g., thresholds). For example, if the normal range of the lab potassium is 3.6 to 5.2, the hospital may only want the program to identify labs that fall at or below 3.2 and above 5.6 if they feel that those are more clinically significant in the care of their patients. Key acute variables are diagnosis that are either comorbid or acute conditions that are associated with an increased risk of morbidity or mortality, such as, for example, hypotension, chronic respiratory failure, DM with hyperglycemia, anemia, chronic kidney disease, hypertension, etc.

Clinical Criteria is the clinical data that is pulled together according to the algorithms from the various areas in the EHR to support the diagnosis that is being recommended by the present invention. This data can be pulled using NLP to pull unstructured data from reports and clinical notes as well as structured data elements from laboratory reports, vital signs, medication lists. Some examples of clinical criteria are blood pressure, blood sugar, white blood cell count, hemoglobin, hematocrit, phrases such as “mass effect”, words such as “atelectasis”, etc. in accordance with embodiments of the present invention.

In some embodiments, the validated results can be stored as EHRs, and postprocessing can be performed using updated patient data to generate updated potential co-morbidities in block 816 in accordance with embodiments of the present invention. The results can be output in block 818 (e.g., sent to GUI, workstation, smartphone, PDA, stored in an EHR, etc.). The method 800 can be iteratively repeated according to user specified customizations (e.g., time search adjustment, threshold number of iterations/results returned/time of search, etc., particular condition (e.g., co-morbidity) found, etc.) in accordance with various embodiments of the present invention.

Referring now to FIG. 9, a block diagram showing a system 900 for automated determination, analysis, and/or presentation of patient co-morbidities is illustratively depicted in accordance with embodiments of the present invention.

In block 902, an input receiving and/or submitting device 902 can be employed to acquire input for analysis/processing in accordance with embodiments of the present invention. A customizer/tuner device 904 can be employed for customizing and/or tuning parameters and/or functionality of the present invention. A pre-processor 906 can be employed for pre-processing patient data for co-morbidities, and a post-processor 912 can be employed for post-processing patient data for updated co-morbidities in accordance with various embodiments of the present invention. A display customizer (e.g., GUI creator) 908 can be employed to create custom interfaces 910 (e.g., e.g., customized GUI, smartphone screen, tablet layout, etc.), and determined, validated co-morbidities, EHRs, GUI layouts, etc. can be stored in a storage device for future use. A natural language/unstructured data processing device 916 can be employed to pull data from unstructured reports (e.g., physician notes, Radiology reports, etc.) for processing by the system 900 in accordance with various embodiments of the present invention.

In the embodiment shown in FIG. 9, the elements thereof are interconnected by a bus 901. However, in other embodiments, other types of connections can also be used. Moreover, in an embodiment, at least one of the elements of system 900 is processor-based and/or a logic circuit. Further, while one or more elements may be shown as separate elements, in other embodiments, these elements can be combined as one element. The converse is also applicable, where while one or more elements may be part of another element, in other embodiments, the one or more elements may be implemented as standalone elements. These and other variations of the elements of system 900 are readily determined by one of ordinary skill in the art, given the teachings of the present principles provided herein, while maintaining the spirit of the present principles.

In accordance with various embodiments, the novel system and method of the present invention can enhance work-flow in the emergency room, or other medical department or facility. The inventive method can be performed at multiple times including at time of emergency room (ED) disposition, such as during admission, during discharge from the emergency room, and/or during discharge from an inpatient facility. The method can be performed multiple times for a single patient, if appropriate. Each time the system is run and/or the method is performed, physician documentation supporting medical decision making can be produced via interpretation of data and findings. This documentation can populate the patient's EHR and be stored for subsequent use.

In some embodiments, more serious problems which are not the reason for a patient's visit to the emergency room may be classified as a secondary diagnosis. This is advantageous not only for the patient for treatment decisions, but also for the hospital facility, enabling it to capture billable actions and document medical decision-making. Advantageously, the inventive system can bring orders of magnitude of efficiency improvements to an important ER process. In particular, the system can accrue significant savings in clinician's time and eliminate documentation errors and omissions. Further, by selecting the clinical data to be displayed and verified based on rules which are also displayed, information overload can be avoided.

In order to assess if automated identification and documentation of comorbid conditions in the ER can lead to improvement of hospital quality data by increasing a patient's relative expected risk of mortality, a random sample of 90 patients who were admitted through an Emergency Department and died during the inpatient encounter were de-identified and their administrative billing data such as age, sex, race and diagnosis codes were then run through the appropriate Vizient risk model calculator. Vizient utilizes a risk adjustment process that uses a logistic regression model constructed by binary outcomes in hospital mortality based on the Diagnostic Related Group (DRG) for which the patient was admitted to the hospital. Vizient currently has 346 models that get updated each year to include the latest administrative data from all academic medical centers. Each patient is put in to a model by main diagnostic related group (DRG) and key acute variables for the mortality are defined by diagnosis with ICD-10 codes that have been strongly associated with that DRG. The models also utilize comorbid conditions defined by AHRQ

In order to correctly assess that patient's severity of illness and risk of mortality, only the coefficients for the ICD-10 codes or conditions that were present at the time of admission to the hospital are taken in to consideration in the UHC risk models. For example, below is the model for adult patients that get assigned to DRG 668, 669 and 670:

Model Results for Adult Model Group # 215
Transurethral Procedures

Model Group: # 215 -(Age >= 18) Transurethral procedures w MCC
(MSDRG 868), Transurethral procedures w CC (MSD RG 669), Transure-
thral procedures w/o CC/MCC (MSDRG 670)
Model Diagnostics: Calculation: Chi-sq = 0.651 Validation: Chi-sq =
1.357, F = 2.083, p = 0.5074
Final: Max VIF = 1.098, Hosmer-Lemeshow = 6.024, p = 0.1105, df = 3,
C = 0.840
Mean Observed = 0.0054, Mean Expected = 0.0054
Cases = 9.474
Model Method = Logistic Regression
Model Results (Significant Predictors)

| Coeff | Explanatory Variable | Coeff | Explanatory Variable |
|---|---|---|---|
| −6.204 | Intercept | 1.637 | Bone/bone marrow |
| | | | secondary malignancies |
| 3.241 | Cancer of Esophagus | 1.478 | CC Malnutrition |
| | (CCS 12) | | |
| 3.810 | Sec Dx Group: Brain/ | 1.459 | CC Coagulopthy |
| | Spinal | | |
| 2.821 | Sec Dx Group: GI | 1.410 | GI/liver secondary |
| | | | malignancies |
| 1.780 | CC Congestive Heart | 1.029 | CC Deficiency Anemias |
| | Failure | | |

As an illustrative embodiment, if a 62-year-old white male is admitted through the emergency room and during his hospitalization has a transurethral procedure, he will be in the DRG 668. If he has ICD-10 codes for the diagnosis of CHF, malnutrition, and esophageal cancer and metastasis he will be assessed in the above risk model. If a variable is present, it receives a 1 and if it is not present in receives a 0. For each of the significant predictors in the model, the coefficient is multiplied by the variable coefficient from the model times the patient value for that variable. The above patient has 4 significant variables, CHF, metastasis, esophageal cancer and malnutrition, with variable coefficients of 1.780, 1.410, 3.241, 1.478, respectively. Using the below formula obtained from UHC, where n=the number of significant variables or comorbid conditions, vari=1 or 0, for whether or not the variable is present in the patient or not and coefficient=the coefficient for the variable, the patients expected probability of mortality can be computed as follows:

$$n$$

$$exp ( intercept + \sum coeffi * vari )$$

$$i = 1$$

$$n$$

$$1 + exp ( intercept + \sum ( coeffient * vari )$$

$$i = 1$$

Using this formula, the patients expected probablity of mortality is 0.846, compared to the overall observed mortalilty of the patients in this DRG which is 0.0054. In the UHC risk model methodology, the patient expected mortality is then translated in to a qualitative scale in order to discern the actual relationship between the patient's expected mortality relative to the actual observed mortality rate of the patients in the DRG model. This qualitative scale gives a description of where a patient stands in terms of the rest of the patients that are in the DRG model in terms of mortality. Patients that are classified with an expected mortality as “well below” are ¼ less than the actual mortality rate of the model where patients that are classified as “well above” are 4 times above, as shown in Table 1, below:

TABLE 1

| Description | Explanation |
|---|---|
| Well Below | Expected mortality <= 0.25*model of observed value |
| Below | Expected mortality < model of observed value and > 0.25* |
| | model observed value |
| Equal to | Expected mortality = model of observed value |
| Above | Expected mortality > model of observed value and < |
| | 4.0*model observed value |
| Well Above | Expected mortality >= 4.0*model of observed value |

Returning to the example of the above patient, the expected probability of the patient mortality in the risk model for that DRG is 0.846 which is more that 4× greater than the overall observed mortality in that group, which is 0.0054.

The 90 randomly selected patients were matched with the risk model appropriate for the DRG identified by the administrative discharge data. The significant comorbid conditions for the DRG were then assigned variables and the probability of this patient's mortality was calculated. This number was then compared to the DRG's mean observed mortality number and an assignment of the patient's relative expected mortality of well below, below, equal, above or well above was made. The same sample was then run through the Emergency Department's comorbidity software and the conditions that were found were compared against the administrative billing data for each patient to determine if it was a newly found comorbid condition not previously documented or coded, or a comorbidity currently found on the coded data but not appropriately identified as being present on admission. All patients with newly identified comorbid conditions or changes in conditions present on admission were reassigned the new coefficients, recalculated and assigned relative risk.

The Emergency Department comorbidity program found one or more significant comorbid conditions in 55 out of the 90 cases (61%). Twenty two cases that had a relative expected mortality of well below or below were changed to above or well above using the software (40%). Eighteen cases went from an expected mortality of above to well above and nine cases had conditions identified that did not make any impact on the relative risk of mortality. Twenty four cases had comorbid conditions that were coded in the administrative data but were not identified as being present on admission (44%). The comorbidity software found 1.06 significant diagnoses in 55 cases, and results are shown in Table 2, below:

TABLE 2

| | | UHC Expected Mortality | UHC Expected | UHC Mean | | | |
|---|---|---|---|---|---|---|---|
| Patient | Coded | for this patient prior | Mortality for | Observed | Relative Risk | Relative risk | |
| # | DRG | to intervention | this Patient | Mortality | Before | after | Add |
| 1 | 441 | 0.4314 | 0.4835 | 0.0676 | well above | well above | malnutrition |
| 2 | 955 | 0.1104 | 0.7584 | 0.2581 | below | above | Hypotension and |
| | | | | | | | comatose |
| 3 | 207 | 0.169103 | 0.169103 | 0.3298 | below | below | |
| 4 | 377 | 0.5163 | 0.719503 | 0.0208 | above | well above | ARF, Acidosis |
| 5 | 64 | 0.3535 | 0.8207 | 0.0967 | above | well above | acidosis |
| 6 | 853 | 0.2673 | 0.2803 | 0.136 | above | above | hypotension |
| 7 | 194 | 0.010836 | 0.010836 | 0.0208 | below | below | |
| 8 | 871 | 0.1482 | 0.1482 | 0.1189 | above | above | |
| 9 | 66 | 0.51597 | 0.6497 | 0.9973 | below | below | hypostension |
| 10 | 189 | 0.0268 | 0.0768 | 0.0027 | equal | well above | mets |
| 11 | 085 (004) | 0.116397 | 0.116397 | 0.0389 | well above | well above | |
| 12 | 189 | 0.1467 | 0.719705 | 0.0768 | above | well above | sepsis |
| 13 | 237 | 0.335762 | 0.861762 | 0.0907 | above | well above | acidosis |
| 14 | 871 | 0.1482 | 0.1833 | 0.1482 | equal | above | chronc resp failure/ |
| | | | | | | | hyponatremia |
| 15 | 856 (004) | 0.0468 | 0.0468 | 0.0149 | above | above | |
| 16 | 834 | 0.7598 | 0.7598 | 0.129769 | well above | well above | |
| 17 | 871 | 0.5958 | 0.744 | 0.1482 | above | well above | AKI |
| 18 | 963 | 0.999 | 0.999 | 0.1096 | well above | well above | |
| 19 | 871 | 0.856 | 0.16 | 0.1482 | above | well above | acidosis hyperuremia, |
| | | | | | | | AKI, Severe sepsis |
| 20 | 64 | 0.0319 | 0.2039 | 0.0967 | | | cerebral compression |
| 21 | 207 | 0.262023 | 0.756023 | 0.3429 | well below | above | hyponatremia, |
| | | | | | | | hypokalemia, |
| | | | | | | | acidosis, ABLA |
| 22 | 308 | 0.992202 | 0.992202 | 0.0165 | well above | well above | |
| 23 | 291 | 0.14475 | 0.25275 | 0.0251 | well above | well above | acute resp failure |
| 24 | 300 | 0.583219 | 0.583219 | 0.0253 | well above | well above | |
| 25 | 813 | 0.0015 | 0.03009 | 0.0261 | well below | well above | acute resp failure |
| 26 | 698 | 0.013 | 0.02175 | 0.0085 | well below | above | THROMBOCYTOPENIA, |
| | | | | | | | DEHYDRATION |
| 27 | 64 | 0.2575 | 0.898803 | 0.0967 | above | well above | coma, shock |
| 28 | 871 | 0.3891 | 0.3891 | 0.1482 | above | above | |
| 29 | 193 | 0.255784 | 0.255784 | 0.0178 | well above | well above | |
| 30 | 871 | 0.1651 | 0.2174 | 0.1482 | above | above | malnutrition |
| 31 | 233 | 0.176 | 0.176 | 0.172 | equal | equal | |
| 32 | 435 | 0.116702 | 0.116706 | 0.0828 | above | above | |
| 33 | 377 | 0.1482 | 0.1659 | 0.1659 | below | equal | SIRS, Shock |
| 34 | 003 (216) | 0.249 | 0.4263 | 0.0728 | below | well above | hypokalemia/hypercalcemia |
| 35 | 23 | 0.127 | 0.37724 | 0.0271 | above | well above | resp fail |
| 36 | 597 | 0.024387 | 0.024387 | 0.0814 | below | below | |
| 37 | 115 | 0.0199 | 0.0769 | 0.0044 | above | well above | AKI, Hypekalemia |
| 38 | 377 | 0.003439 | 0.003439 | 0.0208 | well below | | |
| 39 | 61 | 0.0713 | 0.178286 | 0.0713 | equal | above | |
| 40 | 871 | 0.0677 | 0.1482 | 0.1482 | below | equal | |
| 41 | 917 | 0.923438 | 0.923438 | 0.0227 | well above | well above | |
| 42 | 867 | 0.012567 | 0.8564 | 0.0695 | below | well above | Acute delerium, AKI, |
| | | | | | | | severe sepsis, |
| | | | | | | | coagulopahty, shock, |
| | | | | | | | coma |
| 43 | 237 | 0.0324 | 0.42727 | 0.0907 | below | well above | shock |
| 44 | 829 | 0.799 | 0.779 | 0.0355 | above | above | |
| 45 | 823 | 0.6677 | 0.6677 | 0.66774 | equal | equal | |
| 46 | 811 | 0.00154 | 0.0731 | 0.0052 | below | well above | malnutrtion, Acute |
| | | | | | | | resp fail |
| 47 | 808 | 0.02 | 0.167145 | 0.0206 | equal | well above | malnutrition/acute |
| | | | | | | | resp distress |
| 48 | 871 | 0.01583 | 0.6064 | 0.1482 | well below | well above | Acute resp fail, |
| | | | | | | | Cardiac arrest, coma |
| 49 | 870 | 0.9711 | 0.9711 | 0.1482 | well above | well above | |
| 50 | 85 | 0.623634 | 0.623634 | 0.0389 | well above | well above | |
| 51 | 291 | 0.165619 | 0.21467 | 0.0251 | well above | well above | abla, |
| 52 | 208 | 0.0669 | 0.2055 | 0.2256 | well below | equal | hypotension |
| 53 | 299 | 0.102385 | 0.102385 | 0.0253 | well above | well above | |
| 54 | 871 | 0.014898 | 0.1482 | 0.1521 | well below | below | acute resp distress |
| 55 | 291 | 2.0578 | 2.5436 | 0.251 | well above | well above | thrombocytopenia |
| | | | | | | | POA |
| 56 | 542 | 0.0246 | 0.455 | 0.0446 | below | well above | hyponatrmia, anemia |
| 57 | 955 | 0.5479 | 0.9949 | 0.2581 | above | well above | coma. Cerebreal |
| | | | | | | | edema, cerebral |
| | | | | | | | compression |
| 58 | 871 | 0.1482 | 0.1482 | 0.7501 | below | below | |
| 59 | 193 | 0.033 | 0.427759 | 0.0178 | well below | above | malnutrition/sepsis |
| 60 | 882 | 0.045782 | 0.045782 | 0.0379 | above | above | |
| 61 | 237 | 0.07584 | 0.758413 | 0.0907 | below | well above | CHF |
| 62 | 871 | 0.265 | 0.4509 | 0.1482 | above | above | coma |
| 63 | 545 | 0.192787 | 0.192787 | 0.0292 | well above | well above | |
| 64 | 849 | 0.0025 | 0.0057 | 0.0025 | equal | above | chronic respiratory |
| | | | | | | | failure |
| 65 | 291 | 0.006018 | 0.006018 | 0.0251 | well below | well below | |
| 66 | 237 | 0.127 | 0.293178 | 0.0907 | above | above | hyponat/hypokalemia |
| 67 | 922 | 0.3974 | 0.3974 | 0.0611 | well above | well above | |
| 68 | 823 | 0.00524 | 0.4 | 0.0524 | equal | well above | hyponatremia/ypokalemai |
| 69 | 64 | 0.9153 | 0.9663 | 0.0967 | well above | well above | brain compression |
| | | | | | | | cerebral edema |
| 70 | 216 | 0.682438 | 0.682438 | 0.0728 | well above | well above | |
| 71 | 559 | 0.038431 | 0.129431 | 0.0132 | above | well above | Acute Resp Fail-POA |
| 72 | 871 | 0.0142 | 0.6626 | 0.1482 | well below | well above | acute resp failure |
| 73 | 237 | 0.06111 | 0.293178 | 0.0907 | below | above | hyponatremia/hypokalemia |
| 74 | 981 | 0.26338 | 0.309384 | 0.0085 | well above | well above | AKI, hyponatremia, coma, |
| | | | | | | | DMII, cellulitis, |
| | | | | | | | pleural effusions |
| 75 | 834 | 0.1542 | 0.232366 | 0.1336 | above | well above | shock |
| 76 | 219 (003) | 0.0472 | 0.0472 | 0.0359 | above | above | |
| 77 | 208 | 0.206689 | 0.206689 | 0.0379 | well above | well above | |
| 78 | 003 (237) | 0.060256 | 0.060256 | 0.0907 | below | below | |
| 79 | 374 | 0.099133 | 0.28847 | 0.0722 | above | well above | hyponatremia, hypokalemia, |
| | | | | | | | acidosis, ABLA |
| 80 | 25 | 0.009776 | 0.08875 | 0.0271 | below | above | cerebral compression, |
| | | | | | | | ventriculostomy, |
| | | | | | | | craniectomy, SDH |
| 81 | 25 | 0.142339 | 0.142339 | 0.0271 | well above | well above | |
| 82 | 871 | 0.3351 | 0.3351 | 0.1482 | well above | well above | |
| 83 | 871 | 0.01649 | 0.2577 | 0.1482 | well below | above | acute resp failure |
| 84 | 871 | 0.0162 | 0.402 | 0.1482 | well below | above | hypoxia, hypothermia, |
| | | | | | | | acidosis |
| 85 | 853 | 0.0162 | 0.2763 | 0.136 | well below | above | renal failure POA |
| 86 | 54 | 0.208004 | 0.208004 | 0.0475 | well above | well above | |
| 87 | 177 | 0.761333 | 0.761333 | 0.0379 | well above | well above | |
| 88 | 871 | 0.3037 | 0.4535 | 0.1482 | above | above | SPCM; Acute Resp |
| | | | | | | | Failure |
| 89 | 177 | 0.312813 | 0.312813 | 0.0379 | well above | well above | |
| 90 | 228 | 0.0117876 | 0.2458 | 0.1482 | well below | above | sepsis/malnutition |

The above results of this study not only show that the accuracy of administrative data can be questionable but also sheds light on some of the problems that hospitals will face when moving to a payment system based on quality outcomes. The 40% of cases that went from well below or below in relative mortality risk to above or well above, significantly improves the outcomes data for this hospital. When using the data set for this project and the observed mortality of 90, the expected mortality prior to the use of the comorbidity software was 58, making the observed to expected rate of mortality O/E=1.55. After the improved capture of comorbid conditions with the software, the expected mortality improves to 80, changing the mortality index O/E=1.13. The other significant problem is the 44% of cases that had comorbid conditions that were in the administrative data but were not coded as present on admission. In this new era of pay for performance, it may appear that these conditions were hospital acquired. Conditions such as sepsis, post op respiratory failure, malnutrition that occur during the hospitalization will be no longer be diagnoses that are more highly reimbursed but will be indicators of poor care and the hospital will be penalized.

On average there were only 1.09 diagnoses added to each case to make an impact on the relative mortality risk, yet the impact was tremendous. As hospitals catch on to the importance of the documentation and accurate coding of secondary comorbid conditions, hospitals will put more resources in to these areas and will bend the mortality rate from above the benchmark to below in order to be successful. Thus, currently, it is very difficult to tell whether or not a hospital's quality of care is really slipping or if they just do not have the resources to monitor the administrative data.

Automated documentation of co-morbid conditions (e.g., conditions that can often be overlooked using conventional systems and methods), in accordance with various embodiments of the present invention, can make large improvements in the quality and accuracy of data while reducing rather than adding excessive time requirements to a physician's workload. Expansion of the current list of comorbidities (e.g., during customization) to include those based on non-lab test results, in particular radiology reports is advantageous for identifying other major diagnoses that impact mortality. The use of natural language processing for the analysis of all kinds of text-based reports (EKG reports, radiology reports, etc.) and the presentation of associated images (e.g., within the EKG system) in accordance with various embodiments of the present invention provide significant advantages (e.g., accuracy, speed, reliability, etc.) over any conventional systems and methods.

Having described preferred embodiments of a system and method for automated determination, analysis, and/or presentation of patient co-morbidities (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims.

What is claimed is:

1. A system for identifying and documenting patient co-morbidities and comorbid conditions and thereby decreasing risk-adjusted mortality rates, the system comprising:
a hardware processor operatively coupled to a non-transitory computer-readable storage medium, the processor being configured for:
identifying, using natural language processing (NLP), patient co-morbidity-related clinical data for a patient by preprocessing input historical patient data in real time based on one or more rules, the one or more rules being customizable by tuning threshold values or ranges of one or more of the one or more rules to any of a plurality of user-specified values or ranges;
displaying the identified co-morbidity-related clinical data and the one or more rules using an interactive customizable graphical user interface (GUI) for validation;
accessing and displaying via the GUI the one or more rules on a smartphone device;
accessing further details regarding the rules to increase usability and readability of the system for smartphone devices;
verifying the identified co-morbidity-related clinical data via the GUI prior to storing any of the identified co-morbidity-related clinical data in a remote electronic health record (EHR);
automatically storing the verified results in the remote EHR for the patient;
identifying updated co-morbidity-related clinical data by postprocessing input updated patient data based on the one or more rules;
displaying and validating the updated co-morbidity-related clinical data using the GUI for the validating of the updated co-morbidity-related clinical data; and
detecting changes to the updated and verified co-morbidity data in the remote EHR by the post processing;
iteratively updating the remote EHR in real time with the updated and verified co-morbidity data by the postprocessing upon detection of the changes to the remote EHR, the iterative updating resulting in a reduction of processing requirements and an increase in speed in updating of the remote EHR;
wherein the step of iteratively updating the remote EHR in real time is completed during a patient visit in real time.

2. The system as recited in claim 1, wherein the GUI is customizable for interoperability with any of a plurality of types of electronic health record (EHR) systems.

3. The system as recited in claim 1, wherein the hardware processor is further configured for adjusting time search parameters to any of a plurality of historical time ranges.

4. The system as recited in claim 1, wherein the input historical patient data is in a non-structured format, the non-structured data being processed using a natural language processor.

5. The system as recited in claim 1, wherein the input historical patient data is obtained from a structured report.

6. A method for identifying and documenting patient co-morbidities and comorbid conditions and thereby decreasing risk-adjusted mortality rates, comprising:
identifying, using natural language processing (NLP), patient co-morbidity-related clinical data by preprocessing input historical patient data based on one or more rules, the one or more rules being user-customizable by tuning values or ranges of one or more of the one or more rules to any of a plurality of user-specified values or ranges using an interactive, customizable graphical user interface (GUI);
displaying the identified relevant co-morbidity-related clinical data and the one or more rules using the GUI;
verifying, using a validation feature of the GUI the identified co-morbidity-related clinical data in a remote electronic health record (EHR);
automatically storing, using a non-transitory computer-readable storage medium, verified results in the remote EHR for the patient;
identifying updated co-morbidity-related clinical data by postprocessing input updated patient data based on the one or more rules;
displaying and validating the updated co-morbidity-related clinical data using the GUI, the GUI being configured to improve usability of the system by being customized for readability on a smartphone device display by presenting only respective buttons on the GUI display directed to accessing further information stored on a remote server regarding the relevant co-morbidities, clinical conditions, and corresponding rules from the updated co-morbidity-related clinical data, wherein a particular type of GUI button activation displays further details regarding the relevant co-morbidities, clinical conditions, and corresponding rules from the updated, verified co-morbidity-related clinical data as a pop-up window or on a new display screen to increase usability and readability of the system;
detecting changes to the updated and verified co-morbidity data in the remote EHR by the post processing; and
iteratively updating the remote EHR in real time with the updated, verified co-morbidity data by the postprocessing upon detection of the changes to the remote EHR, the iterative updating resulting in a reduction of processing requirements and an increase in speed in updated of the remote EHR;
wherein the step of iteratively updating the remote EHR in real time is completed during a patient visit in real time.

7. The method as recited in claim 6, wherein the GUI is customizable for interoperability with any of a plurality of types of electronic health record (EHR) systems.

8. The method as recited in claim 6, further comprising adjusting time search parameters to any of a plurality of time ranges.

9. The method as recited in claim 6, wherein the input historical patient data is in a non-structured format, the non-structured data being processed using a natural language processor.

10. The method as recited in claim 6, wherein the input historical patient data is obtained from a structured report.

11. A non-transitory computer readable storage medium comprising a computer readable program for identifying and documenting patient co-morbidities and comorbid conditions and thereby decreasing risk-adjusted mortality rates, wherein the computer readable program when executed on a computer causes the computer to perform the steps of:
identifying, using natural language processing (NLP), patient co-morbidity-related clinical data by preprocessing input historical patient data based on one or more rules, the one or more rules being customizable by tuning values or ranges of one or more of the one or more rules to any of a plurality of user-specified values or ranges;
displaying the identified relevant co-morbidity-related clinical data and the one or more rules using an interactive customizable graphical user interface (GUI) for verification;
accessing and displaying, via the GUI, the one or more rules on a smartphone device;
in response to a user action on the smartphone device, displaying further details regarding the rules or the button as a pop-up window or on a new display screen to increase usability and readability of the system,
verifying the identified co-morbidity-related clinical data via the GUI prior to storing any of the identified co-morbidity-related clinical data in a remote electronic health record (EHR);
automatically storing, using a non-transitory computer-readable storage medium, verified results in the remote EHR for the patient;
identifying updated co-morbidity-related clinical data by postprocessing input updated patient data based on the one or more rules;
displaying and validating the updated co-morbidity-related clinical data using the GUI for verifying of the updated co-morbidity related clinical data, the GUI being configured for improving usability of the system by being customized for readability on a smartphone device by presenting respective buttons on the GUI display directed only to accessing further information stored on a remote server regarding the relevant co-morbidities, clinical conditions, and corresponding rules from the updated verified co-morbidity-related clinical data;
detecting changes to the updated and verified co-morbidity data in the remote EHR by the post processing; and
iteratively updating the remote EHR in real time with only the updated, verified co-morbidity data by the postprocessing upon detection of the changes to the EHR based on the verifying the updated co-morbidity-related clinical data, the iterative updating resulting in a reduction of processing requirements and an increase in speed in updating of the remote EHR;
wherein the step of iteratively updating the remote EHR in real time is completed during a patient visit in real time.

12. The system as recited in claim 1, wherein hospital-acquired conditions for individual patients are automatically monitored and detected based on the iteratively updating the remote EHR with the identified updated co-morbidity data by the postprocessing upon detection of changes to the remote EHR in real-time.

13. The system as recited in claim 1, wherein multiple fields within the EHR are searchable with a single user action on the GUI, the single action being executable by an on-screen button push, a long-press, or a short-press with the results appearing in a pop-up window displayed in front of the GUI buttons on the display.

14. The method as recited in claim 6, wherein GUI is configured for the tuning values or ranges of one or more of the one or more rules and the verification in real-time by an end user using one or more customizable GUI buttons.

15. The system as recited in claim 1, wherein the GUI is customized for readability on a smartphone device display by presenting respective graphical buttons on the GUI display directed only to accessing further information stored on a remote server regarding the relevant co-morbidities, clinical conditions, and corresponding rules from the updated co-morbidity-related clinical data, wherein a particular type of graphical button activation displays further details regarding the relevant co-morbidities, clinical conditions, and corresponding rules from the updated, verified co-morbidity-related clinical data as a pop-up window or on a new display screen to increase usability and readability of the system.

16. The method as recited in claim 6, wherein the GUI is configured for accessing and displaying the one or more rules by a particular type of graphical button activation on a smartphone device, wherein the particular type of graphical button activation accesses further details regarding the rules or the button as a pop-up window or on a new display screen to increase usability and readability of the system.

17. The non-transitory computer readable storage medium as recited in claim 11, wherein the GUI is configured for accessing and displaying the one or more rules by a particular type of graphical button activation on a smartphone device, wherein the particular type of graphical button activation accesses further details regarding the rules or the button as a pop-up window or on a new display screen to increase usability and readability of the system.
